(19) United States
(12) Patent Application Publication
(10) Pub. No.: US 2024/0269135 A1
(43) Pub. Date: Aug. 15, 2024

(54) METHODS OF TREATING BREAST CANCER

(71) Applicant: ASTRAZENECA AB, SODERTALJE (SE)

(72) Inventor: ANITRA FIELDING, WILMINGTON, DE (US)

(21) Appl. No.: 18/564,778

(22) PCT Filed: May 31, 2022
(86) PCT No.: PCT/EP2022/064697

§ 371 Date: Nov. 28, 2023

Related U.S. Application Data
(60) Provisional application No. 63/195,795, filed on Jun. 2, 2021.

(51) Int. Cl. A61K 31/502; A61P 35/00

(52) U.S. Cl. CPC A61K 31/502; A61P 35/00

(57) ABSTRACT

This disclosure relates to methods for the adjuvant treatment of a subject having germline mutated BRCA1 and/or BRCA2 breast cancer, wherein the subject has previously received local treatment for the breast cancer. and neoadjuvant or adjuvant chemotherapy.

### BACKGROUND OF THE DISCLOSURE

### Field of the Disclosure

This disclosure relates to adjuvant treatment methods for subjects having HER2-negative, BRCA1 and/or BRCA2 germline gene mutated breast cancer, who have previously received local treatment (e.g. surgery to remove breast tissue) and neoadjuvant or adjuvant chemotherapy.

### BACKGROUND

Poly(ADP-ribose)-polymerase inhibitors target cancers with homologous-recombination-repair defects by synthetic lethality. Novel therapies are needed to reduce recurrence in patients with BRCA1/2 germline mutation-associated early breast cancer.

### SUMMARY

In an aspect, the present specification describes a method of preventing, reducing, or delaying the reoccurrence of breast cancer in a subject following local treatment and neoadjuvant or adjuvant chemo therapy, the method comprising:
administering to the subject a therapeutically effective amount of 4-[(3-{[4-(cyclopropane carbonyl)piperazine-1-yl]carbonyl}-4-fluorophenyl)methyl]-2H-phthalazin-1-one (olaparib), or a hydrate, solvate, or prodrug thereof.

In a further aspect, the present specification describes a method of treating a subject with breast cancer following local treatment and neoadjuvant or adjuvant chemo therapy, said method comprising the adjuvant treatment of the subject with a therapeutically effective amount of 4-[(3-{[4-(cyclopropane-carbonyl)piperazine-1-yl]carbonyl}-4-fluorophenyl)methyl]-2H-phthalazin-1-one (olaparib), or a hydrate, solvate, or prodrug thereof.

In a further aspect, the present specification describes 4-[(3-{[4-(cyclopropane carbonyl)piperazine-1-yl]carbonyl}-4-fluorophenyl)methyl]-2H-phthalazin-1-one (olaparib), or a hydrate, solvate, or prodrug thereof for use in (or for use in the manufacture of a medicament for) the adjuvant treatment, after local treatment and neoadjuvant or adjuvant chemotherapy, of a subject having breast cancer.

In a further aspect, the present specification describes a method of improving invasive disease survival (or overall survival or distant-disease-free survival) by providing adjuvant treatment to a subject with a prior diagnosis of HER2-negative germline mutated BRCA1 and/or BRCA2 breast cancer, said subject previously having had local treatment (e.g. surgery, such as surgery to remove diseased breast tissue) and neoadjuvant or adjuvant chemotherapy, the method comprising the step of administering to such a subject a therapeutically effective amount of 4-[(3-{[4-(cyclopropane-carbonyl)piperazine-1-yl]carbonyl}-4-fluorophenyl)methyl]-2H-phthalazin-1-one (olaparib), or a hydrate, solvate, or prodrug thereof.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 Shows the Kaplan-Meir estimates of survival of subjects receiving either adjuvant olaparib therapy or placebo. Panel (A) shows invasive-disease-free survival (IDFS). Panel (B) shows distant-disease-free survival. Panel (C) shows overall survival (OS).

FIG. 2 shows a subgroup analysis of Invasive Disease-free Survival.

### DETAILED DESCRIPTION OF THE DRAWINGS

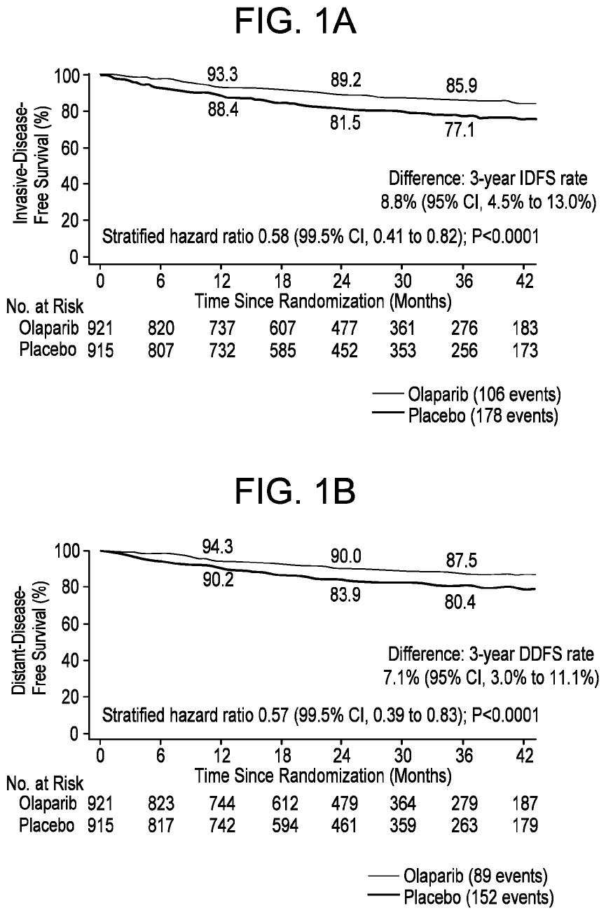

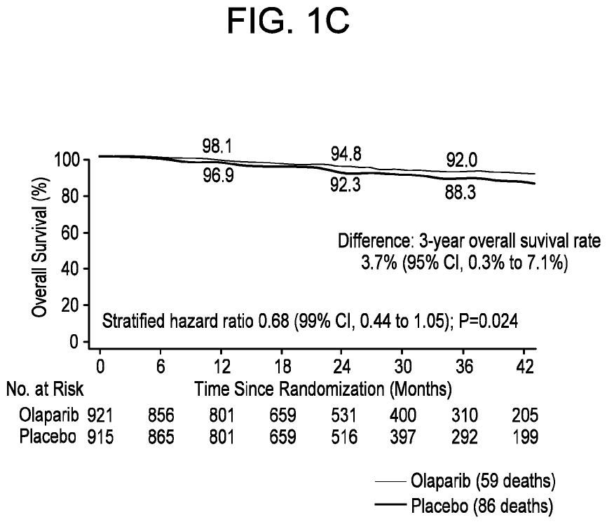

FIG. 1. Kaplan-Meier Estimates of Survival: in accordance with the STEEP system the primary endpoint of invasive-disease-free survival (Panel A) is defined as the time from randomization until the date of one of the following events: ipsilateral invasive breast tumor; locoregional invasive disease; distant recurrence; contralateral invasive breast cancer; second primary invasive cancer; or death from any cause. Patients without documented invasive-disease-free survival event were censored at the date they were last known to be disease free.

Distant-disease-free survival (Panel B) is defined as the time from randomization until documented evidence of first distant recurrence of breast cancer or death. Distant recurrence includes the following events: distant recurrence (metastatic disease-breast cancer that has either been biopsy confirmed or radiologically diagnosed as recurrent invasive breast cancer); death attributable to any cause, including breast cancer, non-breast cancer, or unknown cause; second primary non-breast invasive cancer. Evidence of distant recurrence requires either radiological examination or histopathological confirmation by biopsy.

Overall survival (Panel C) is defined as the time from the date of randomization until death due to any cause. The P value for the boundary for significance in this prespecified event-driven interim analysis was <0.01.

99.5% confidence intervals are shown for the hazard ratios for invasive-disease-free survival and distant-disease-free survival because P<0.005 is required to indicate statistical significance for these end points. Similarly, the 99% confidence intervals are shown for the hazard ratio for overall survival because P<0.01 is required to indicate statistical significance for overall survival.

On the basis of the pooling strategy for stratification factors described in the Supplementary Appendix Section 3.3, both the Cox model hazard ratio estimation and the log rank test were performed with hormone receptor status as the single stratification factor.

The event-free rates at 12, 24, and 36 months in each arm are displayed above and below the curves.

CI denotes confidence interval, DDFS distant-disease-free survival, IDFS invasive-disease-free survival.

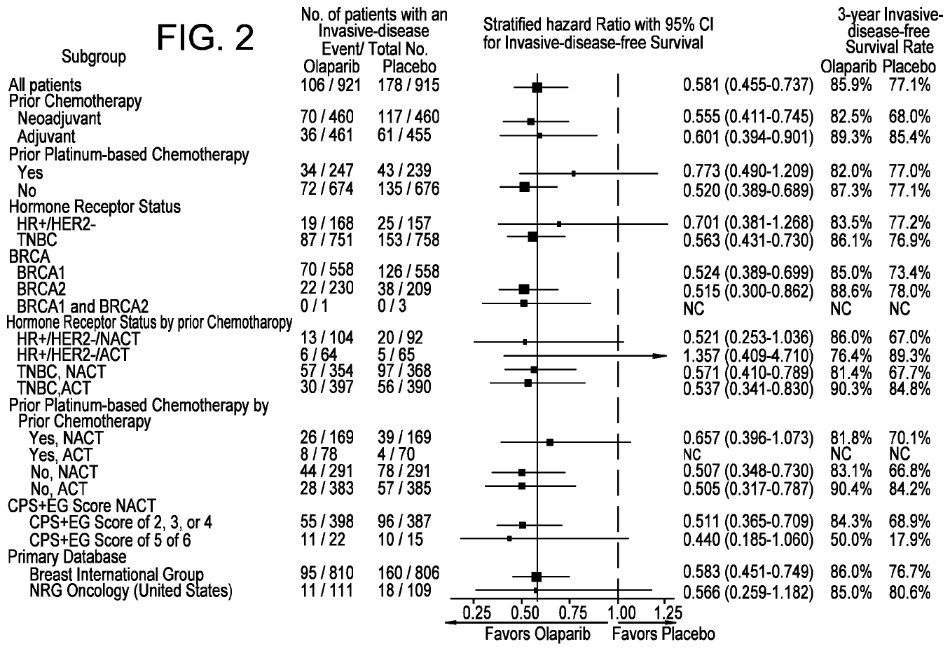

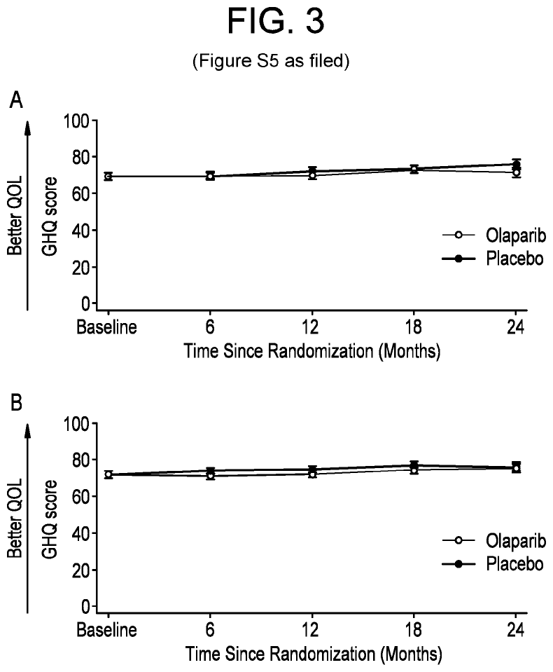

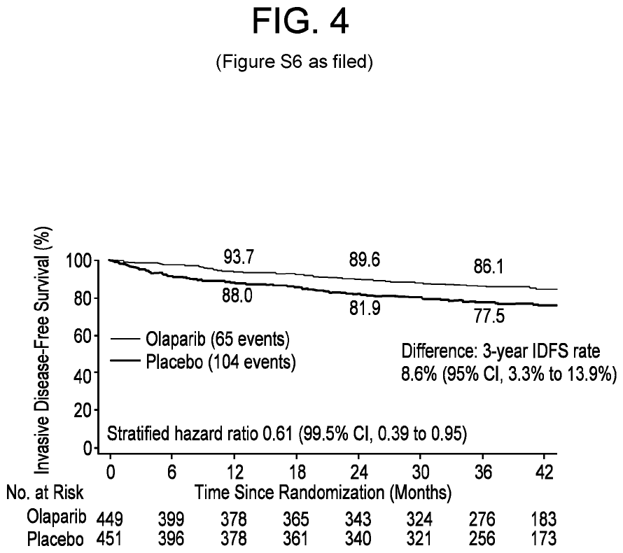

FIG. 2. Subgroup Analysis of Invasive Disease-free Survival: the solid vertical line indicates the overall hazard ratio estimate and the dashed vertical line indicates hazard ratio of 1.00, as recommended by Cuzick.²³ The size of the blue squares corresponds to the number of events contributing to the estimate of the treatment effect (i.e., proportional to square root of 1/variance of the estimated hazard ratio). Even without correcting for multiple comparisons none of the tests for heterogeneity reached statistical significance. The CPS&EG score is a staging system for disease specific survival in patients with breast cancer treated with neoadjuvant chemotherapy.²⁰ This incorporates pretreatment clinical stage, estrogen receptor status, nuclear grade and post-neoadjuvant chemotherapy pathological stage. ACT denotes adjuvant chemotherapy; HER2 denotes human epidermal growth factor receptor 2; HR+ denotes hormone-receptor-positive; NACT denotes neoadjuvant chemotherapy; TNBC denotes triple-negative breast cancer.

*Pre-specified subgroup analysis. Includes all patients that received neoadjuvant chemotherapy, whether they have hormone receptor positive or triple-negative disease.

### DETAILED DESCRIPTION OF THE DISCLOSURE

As used herein, the term “about” when referring to any given numerical value means within +10%, +5%, or +2% of that value.

The methods of the disclosure also require administration of olaparib. As used herein, “olaparib” refers to 4-[(3-{[4-(cyclopropane-carbonyl)piperazine-1-yl]carbonyl}-4-fluorophenyl)methyl]-2H-phthalazin-1-one, or a hydrate, solvate, or prodrug thereof. 4-[(3-{[4-(cyclopropane-carbonyl)piperazine-1-yl]carbonyl}-4-fluorophenyl)methyl]-2H-phthalazin-1-one, having the following structure, is disclosed in International Publication No. WO 2004/080976 A1, incorporated by reference herein.

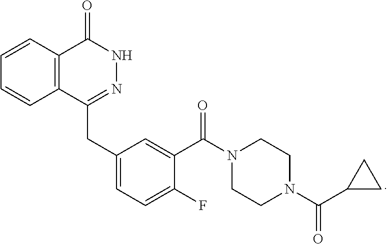 [SMILES: O=C(c1cc(Cc2n[nH]c(=O)c3ccccc23)ccc1F)N1CCN(C(=O)C2CC2)CC1]

Olaparib is administered preferably in the form of a pharmaceutical composition. The therapeutically effective amount of olaparib has been previously established. As described herein, the therapeutically effective amount of olaparib is in the range of about 400 to 800 mg per day. For example, in certain methods described herein, olaparib is administered in an amount of about 600 mg daily (e.g., about 300 mg taken twice daily).

### EXAMPLES

Poly(ADP-ribose)-polymerase inhibitors target cancers with homologous-recombination-repair defects by synthetic lethality. Novel therapies are needed to reduce recurrence in patients with BRCA1/2 germline mutation-associated early breast cancer.

We conducted a randomized double-blind phase 3 trial involving patients with HER2-negative early breast cancer with BRCA1/2 germline pathogenic/likely pathogenic variants (gBRCA-P/LP-variant), and high-risk clinico-pathological factors, after local treatment and (neo)adjuvant chemotherapy. Patients were randomly assigned (1:1) to 1 year of oral olaparib or placebo. The primary endpoint was invasive disease-free-survival.

1836 patients were randomized. At pre-specified event-driven interim-analysis with median follow-up 2.5 years, 3-year invasive-disease-free survival 85.9% in the olaparib-group and 77.1% with placebo (difference 8.8%; 95% CI 4.5%, 13.0%; hazard ratio (HR) for invasive-disease-free survival 0.58; 99.5% confidence interval (CI), 0.41, 0.82; p<0.0001). The 3-year distant-disease-free survival 87.5% in the olaparib-group and 80.4% with placebo (difference 7.1%; 95% CI 3.0%, 11.1%; HR for distant-disease-free survival 0.57; 99.5% CI 0.39, 0.83; p<0.0001). Olaparib was associated with fewer deaths than placebo (59 versus 86); HR for overall-survival was 0.68 (99% CI 0.44, 1.05, p=0.024), not statistically significant at an interim-analysis boundary of p<0.01. Safety data were consistent with known toxicities of olaparib with no excess serious adverse events or adverse events of special interest.

Among patients with high-risk, HER2-negative early breast cancer and gBRCA-P/LP-variants, adjuvant olaparib after completion of local treatment and (neo)adjuvant chemotherapy, significantly improves both invasive disease-and distant-disease-free survival with limited impact on global patient-reported quality of life (OlympiA NCT02032823, BIG 6-13, NSABP B-55).

In an aspect, the improvement in invasive disease free survival in patients treated with olaparib at about 3 years is up to about 10%, such as up to about 9%, such as up to about 8%, such as from about 1 to about 9%, such as from about 1 to about 8%, such as from about 5% to about 10%, such as from about 5% to about 9%. In an aspect, the improvement in invasive disease free survival in patients treated with olaparib at about three years is about 9%. In an aspect, the improvement in invasive disease free survival in patients treated with olaparib at three years is about 9%.

In an aspect, the improvement in distant disease free survival in patients treated with olaparib at about 3 years is up to about 8%, such as up to about 7%, such as from about 1 to about 8%, such as from about 1 to about 7%, such as from about 3% to about 8%, such as from about 3% to about 7%. In an aspect, the improvement in distant disease free survival in patients treated with olaparib at about three years is about 7%. In an aspect, the improvement in distant disease free survival in patients treated with olaparib at three years is about 7%.

In an aspect, the improvement in overall survival in patients treated with olaparib at about three years is about 4%. In an aspect, the improvement in overall survival in patients treated with olaparib at three years is about 4%.

Approximately 5% of unselected patients with breast cancer carry a germline BRCA1 or BRCA2 pathogenic/likely pathogenic (P/LP) mutation now termed variants (gBRCA-P/LP-variant).^{1.2}

Such variants are more likely in patients who have a strong family history of breast cancer, are younger, and in those with synchronous or metachronous contralateral breast and ovarian cancer3 or from ethnic groups with known founder variants. 1.2 Patients with a BRCA1-P/LP-variant are particularly pre-disposed to triple-negative (i.e., human epidermal growth factor receptor type 2 [HER2]-negative, estrogen-receptor-negative, and progesterone-receptor-negative) breast cancer (TNBC), whereas patients with a BRCA2-P/LP-variant often develop estrogen-receptor-positive tumors.⁴⁻⁶ Germline testing for such variants is currently performed selectively in such breast cancer patients.⁷

BRCA1 and BRCA2 encode proteins critical for homologous-recombination-DNA-repair.8 Breast cancers with gBRCA-P/LP-variants and biallelic inactivation show evidence of homologous-recombination-deficiency.9,10 Inhibitors of the PARP family of enzymes exploit the principle of synthetic lethality to selectively kill tumor cells¹¹⁻¹⁴ with homologous-recombination-deficiency. Proof of concept for clinical activity was demonstrated in advanced gBRCA-P/LP variant-associated breast, ovarian, prostate and pancreatic cancers¹⁵⁻¹⁷ that justified randomized study designs. In the OlympiA trial, which is described in detail herein, we hypothesized that olaparib would provide benefit as an adjuvant therapy for patients with gBRCA-P/LP variant-associated early breast cancer who have high recurrence risks despite standard of care local and systemic therapy.^{18,19}

### Methods

### Trial Design and Oversight

The trial was designed and conducted as a collaborative partnership between the Breast International Group (BIG) and the sponsors NRG Oncology in the United States (US) and AstraZeneca (AZ) outside the US. OlympiA is a prospective, randomized, multicenter, multinational, double-blind, placebo-controlled clinical trial with eligible patients randomly assigned to receive 1 year of treatment with 300 mg olaparib twice daily or matching placebo following completion of standard (neo)adjuvant chemotherapy and local therapy (Fig.S1: Trial Schema in the Supplementary Appendix).

The trial recruited patients in 420 centers across 23 countries (Table S1 in the Supplementary Appendix).

### Patients and Eligibility Criteria

Patients eligible for the trial harbored a gBRCA-P/LP variant defined by local or central testing and high-risk, HER2-negative primary breast cancer following definitive local treatment and neoadjuvant or adjuvant chemotherapy. If a local laboratory had reported an eligible gBRCA-P/LP variant, this was used for establishing eligibility. Details of gBRCA-P/LP variant screening, local and central gBRCA-P/LP variant testing, and concordance is provided in Figure S2 and Table S2/S3 in Supplementary Appendix. Any gBRCA-P/LP variant eligibility adjudication was conducted by the trial Genetics Advisory Committee. Local estrogen-receptor, progesterone-receptor and HER2 testing results were used for determination of the hormone-receptor status (cut-point for positive was ≥1%) for stratification and for hormone-receptor-positive specific stage criteria for eligibility (details for receptor status central review and concordance for all patients recruited outside China are provided in Supplementary Appendix Tables S4 and S5).

Patients were required to have completed all local therapy including radiotherapy, which interacts with PARP inhibition, at least 2 and not more than 12 weeks before study entry. Patients had completed at least 6 cycles of neoadjuvant or adjuvant chemotherapy containing anthracyclines, taxanes or both agents. Platinum chemotherapy was allowed. Adjuvant bisphosphonates and adjuvant endocrine therapy in patients with hormone-receptor positive disease were given according to institutional guidelines. No chemotherapy after surgery was allowed in patients who received neoadjuvant chemotherapy. Patients with triple-negative breast cancer treated with adjuvant chemotherapy were required to have axillary node-positive disease or an invasive primary tumor pathological size ≥2 cm. Patients treated with neoadjuvant chemotherapy were required to have residual invasive breast cancer in the breast or resected lymph nodes (no pathologic complete response from neoadjuvant therapy).

Patients treated with adjuvant chemotherapy for hormone receptor positive, HER2-negative breast cancer were required to have ≥4 pathologically confirmed positive lymph nodes. Those treated with neoadjuvant chemotherapy were required to have not achieved pCR with a CPS&EG score ≥3 (a combined scoring system to estimate relapse probability based on clinical and pathological stage [CPS] and estrogen receptor status and histologic grade [EG]).²⁰ Full eligibility criteria are in the Supplementary Appendix Section 3.2.

### Randomization and Treatment

Patients were randomized in a 1:1 ratio to 52 weeks treatment with 300 mg olaparib or matching placebo tablets taken orally twice daily.

Patients were stratified by hormone-receptor status (positive versus negative), NACT versus ACT and use of platinum chemotherapy for current breast cancer (yes versus no).

### Assessments

Following randomization, medical history and physical examination were performed on a 4-weekly basis for 24 weeks and then 3-monthly through year 2, 6-monthly assessments in years 3 to 5 and annually thereafter. Imaging to assess development of metastatic disease was obtained at investigator discretion when symptoms, exam or laboratory findings suggested the possibility of disease recurrence. Patients had mammogram and/or breast magnetic-resonance-imaging annually.

After a first event, patients were followed for first distant relapse (if not the first event), CNS metastases, loco-regional relapses, contralateral breast cancer, second primary malignancies and survival status.

### Statistical Analysis

In accordance with the standardized definitions for efficacy end points (STEEP) system,²¹ the primary endpoint of invasive disease-free survival was defined as the time from randomization until the date of first occurrence of one of the following events: ipsilateral invasive breast tumor, locoregional invasive disease, distant recurrence, contralateral invasive breast cancer, second primary invasive cancer or death from any cause. Patients without a documented invasive disease-free survival event were censored at the date they were last known to be disease-free.

Efficacy analyses were based on the intention-to-treat (ITT) population. Survival functions were estimated by Kaplan-Meier method. The stratified Cox proportional-hazards model was used to estimate the hazard ratio and confidence intervals, the comparison of survival between treatment arms was tested by stratified log-rank test. Because of the early period where the hazard ratio was very low, the Cox assumption was not confirmed. According to our statistical analysis plan, restricted mean survival time was calculated and supported the results obtained from the Cox model analysis. Safety was assessed in the population who received at least one dose of study medication.

The study was designed with a sample size of 1800 patients such that the primary analysis would be triggered by 330 invasive disease-free survival events in the ITT population, to achieve 90% power to detect a hazard ratio (HR) of 0.7 assuming a two-sided 5% significance level. A single interim-analysis of the ITT population was planned when 165 invasive disease-free survival events had been observed in the first 900 patients enrolled (the mature cohort). At interim-analysis, an analysis of this mature cohort was also prespecified requiring a HR of similar magnitude to provide confidence in the sustainability of the ITT result. The secondary analyses included distant disease-free survival, overall survival, and safety. To control the type-1 error rate at interim-analysis, superiority boundaries based on a hierarchical multiple testing procedure 22 were p<0.005 for invasive disease-free survival, followed by p<0.005 for distant disease-free survival and p<0.01 for overall survival with confidence intervals for HRs selected to match the required significance levels for each endpoint at the interim-analysis (see Figure S3 in the Supplementary Appendix).

### Results

### Patients

From June 2014 through May 2019, 1836 patients were randomly assigned to receive olaparib or placebo. At the data cut-off on 27Mar. 2020, 284 (86%) of the 330 primary analysis target invasive disease-free survival events had been observed with a median follow-up of 2.5 years (IQR range, 1.5 to 3.5) in the ITT population, and 3.5 years (IQR range, 2.9 to 4.1) in the mature cohort. After randomization, 10 patients in the olaparib-group and 11 patients in the placebo-group did not receive assigned therapy (Figure S4: Consort Diagram in the Supplementary Appendix). Baseline characteristics of the patients were balanced between the two treatment groups (Table 1, and Table S6 in the Supplementary Appendix). 82.2% of the patients had triple-negative breast cancer (hormone receptor and HER-2 negative). Half of patients received adjuvant chemotherapy and half neoadjuvant chemotherapy with the majority (93.7%) receiving an anthracycline-and taxane-containing regimen. A platinum agent was received by 26.5%, primarily in the neoadjuvant setting. gBRCA-P/LP-variants were present in BRCA1 in 72.3% and BRCA2 in 27.2% of patients with an even distribution between treatment groups.

### Efficacy

The early reporting efficacy boundary was crossed at the prespecified interim analysis. The percentage of patients alive and free of invasive disease at 3 years was 85.9% in the olaparib-group and 77.1% in the placebo-group (8.8% difference; 95% confidence interval 4.5%, 13.0%). Invasive disease-free survival was significantly longer in patients randomized to olaparib than to placebo (HR, 0.58; 99.5% confidence interval [CI], 0.41 to 0.82; P<0.0001) (FIG. 1A). Invasive disease-free survival events were reported in 106 and 178 patients in the olaparib and placebo-groups, respectively. The event frequency at all sites was lower with olaparib treatment (Supplementary Appendix Table S7).

Distant disease-free survival at 3 years was 87.5% in the olaparib-group and 80.4% in the placebo-group (7.1% difference; 95% CI 3.0%, 11.1%), significantly longer in patients who received olaparib (HR, 0.57; 99.5% CI, 0.39 to 0.83; P<0.0001) (FIG. 1B).

Fewer deaths were reported in the olaparib-group (n=59) compared to placebo (n=86) with an overall survival HR for death of 0.68; 99% CI, 0.44 to 1.05, p=0.024 (FIG. 1C), which did not cross the prespecified multiple-testing procedure significance boundary of p<0.01 (Figure S3 in the Supplementary Appendix).

Primary cause of death was breast cancer in 93.2% of the olaparib-group and 95.3% of the placebo-group (Table S8 in the Supplementary Appendix). Death without a previous invasive disease-free survival event was reported in 2 patients, both in the olaparib-group (one cardiac arrest, one unknown cause; Table S7 in the Supplementary Appendix).

None of the pre-specified sensitivity analyses, described in Section 3.5 Supplementary Appendix, changed the conclusions reported here (Table S9 Supplementary Appendix).

Subgroup analysis of invasive disease-free survival revealed point estimates of treatment effect for olaparib over placebo consistent with that of the overall analysis population across all the stratification groups and pre-specified subgroups (FIG. 2: Table S10 in the Supplementary Appendix). The benefit of adjuvant olaparib relative to placebo was observed for invasive disease-free survival irrespective of the P/LP-variant being in BRCA1 versus BRCA2, the hormone receptor status, or adjuvant versus neoadjuvant chemotherapy context with confidence intervals that cross the point estimate of the HR for invasive disease-free survival in the overall population.²³ No evidence suggested statistical heterogeneity in the treatment effect across subgroups.

### Safety

A total of 1815 patients (911 in the olaparib-group and 904 in the placebo-group) were included in the safety analysis. The median number of days at 300 mg twice-daily protocol dose was 338 with percentage of intended dose being 94.8% in the olaparib-group, and 358 days and 98.9% in the placebo-group (Tables S11 to S13 in the Supplementary Appendix). Early treatment discontinuations, including discontinuations due to recurrence, occurred in 236 (25.9%) of the olaparib-group and 187 (20.7%) of the placebo-group (Figure S4 in the Supplementary Appendix).

Adverse events occurring in greater than 10% of patients are provided in Table 2 and were consistent with product label. Important adverse events are summarized in Table 3. Adverse events of grade 3 or higher occurring in more than 1% of patients were anemia (8.7%), neutropenia (4.8%), leukopenia (3.0%), fatigue (1.8%) and lymphopenia (1.2%) all in the olaparib-group. Blood transfusion was infrequently required with 5.8% of patients having at least one blood transfusion in the olaparib group compared to 0.9% in the placebo group, with the majority having only one transfusion (4.1%) (Table S14 in Supplementary Appendix). Serious adverse events occurred in 79 patients (8.7%) who received olaparib and 76 patients (8.4%) who received placebo. Adverse events leading to death were cardiac arrest in one patient on olaparib and acute myeloid leukemia (AML) and ovarian cancer in one patient each on placebo. Adverse events of special interest included pneumonitis, radiation pneumonitis, myelodysplastic syndrome (MDS)/AML, and new primary malignancy other than AML/MDS. None were increased by olaparib but, given the short median follow-up of 2.5 years for this report, further follow-up is needed for the latter two adverse event of special interest groups.

In the olaparib-group, 228 patients (25.0%) required a dose reduction compared to 47 (5.2%) in the placebo-group. Adverse events requiring permanent discontinuation of the trial drug occurred in 90 patients (9.9%) in the olaparib-group and 38 patients (4.2%) in the placebo-group. Most common reasons for discontinuation of olaparib were nausea (2.0%), anemia (1.8%), fatigue (1.3%) and neutrophil count decreased (1%). (Table S15, S16 in the Supplementary Appendix). The results of the EORTC QLQ-C-30 Global Health Status/Quality-of-Life scale indicate that global health quality did not decline during the 12 months of treatment with either olaparib or placebo. Any differences between the treatment arms are not considered clinically significant (Figure S5 in the Supplementary Appendix).

### Discussion

Olaparib and talazoparib are now approved for the treatment of metastatic gBRCA-P/LP variant-associated breast cancer following evidence of progression-free-survival benefit, improved tolerability and quality of life compared to standard chemotherapy.^{24,25}

OlympiA was designed to test the efficacy of adjuvant PARP inhibitor therapy with olaparib in patients with early breast cancer and impaired BRCA1 or BRCA2 homologous-recombination function, identified using presence of a BRCA1 or BRCA2 P/LP germline variant as a patient selection biomarker. This trial shows that olaparib given for 52 weeks as adjuvant therapy after (neo)adjuvant chemotherapy and local therapy significantly improves invasive-and distant-disease-free survival in such patients. No prior evidence suggests a differential PARP inhibitor treatment effect related to BRCA1 versus BRCA2 status or hormone-receptor status.^{15,24-26} We find no evidence of heterogeneity, and confidence intervals for hazard ratios in these and other subgroups include the point estimate for the treatment effect seen in the overall population.

The pre-specified interim analysis was timed based on having sufficient events in a mature cohort to provide confidence that treatment effects observed early at interim-analysis in the ITT population would likely be sustained. The evidence of olaparib treatment effect in this mature cohort is reassuring (Figure S6 in the Supplementary Appendix).

Platinum-containing chemotherapy is not considered to be the standard of care in neoadjuvant or adjuvant chemotherapy in HER2-negative early breast cancer.^{27,28} Platinum chemotherapy use was included as a stratification factor because platinum-induced DNA adducts are repaired by homologous-recombination DNA repair and platinum is known to have a specific interaction with gBRCA-P/LP variants in metastatic breast cancer.^{29,30} As with other subgroup analyses, the test for heterogeneity indicated no evidence that olaparib is less effective in patients treated with platinum-based adjuvant or neoadjuvant chemotherapy.

Fewer deaths occurred among patients treated with olaparib than placebo, although at this early timepoint the difference did not achieve the threshold for statistical significance in the pre-specified multiple testing procedure. Longer blinded follow up is required to assess the impact of olaparib on overall survival.

The safety profile of olaparib was consistent with that previously reported; adverse events with olaparib treatment were largely grade 1 or 2. The only grade 3 toxicity occurring in more than 5% of patients was anemia (8.7%), which infrequently required transfusion. Dose interruptions and reductions appear to be effective management strategies. Serious adverse events were also not increased by olaparib. Although PARP inhibitors are DNA interacting drugs,³¹ and have the potential to induce mutation in DNA and hematological malignancies,³² these were not increased by olaparib and further blinded follow-up is continuing.

The selection of a particularly high recurrence risk hormone-receptor-positive population was driven by regulatory concern that low invasive-disease-free survival event rates might not justify exposure to the potential MDS/AML risks perceived for olaparib. Patients with gBRCA-P/LP-variants form a high recurrence risk group who more often require chemotherapy in addition to endocrine therapy,^{18,19} and comprised 14% of those with hormone-receptor-positive HER2-negative breast cancer treated with neoadjuvant chemotherapy in a recent study.³³ A high risk of recurrence was observed in OlympiA where 23% of patients in the hormone-receptor positive population treated with placebo are estimated to have an invasive disease-free survival event within 3 years (FIG. 2). Olaparib treatment administered with endocrine therapy (Table S17 in the Supplementary Appendix) was both safe and effective with no differential treatment effect in this subgroup consistent with the results of other studies both in metastatic and early breast cancer contexts.²⁴⁻²⁶

Patients with triple-negative breast cancer do not currently have any approved adjuvant targeted therapy. Based on the results of the CREATE-X trial, patients with triple-negative breast cancer and residual invasive cancer following neoadjuvant chemotherapy are increasingly treated with post-neoadjuvant capecitabine chemotherapy. This trial did not examine post-neoadjuvant capecitabine effects in patients with gBRCA-P/LP variants, likely to be less than 15% of those accrued.³⁴ Post-neoadjuvant capecitabine was not permitted in OlympiA, as this was not standard-of-care when the study was designed, and so the study cannot inform the relative efficacy of olaparib versus capecitabine in this setting. However, Robson et al²⁴ demonstrated that olaparib is more effective than chemotherapy in prolonging progression-free-survival in metastatic HER2-negative breast cancer patients with gBRCA-P/LP variants in a study where 45% received capecitabine as the comparative therapy.^{24,35}

OlympiA demonstrates that one year of adjuvant olaparib can meaningfully reduce recurrence risk and prevent progression to metastatic disease in patients with high-risk early breast cancer and gBRCA-P/LP variants with high adherence rates and primarily a low-grade toxicity profile. Patients with gBRCA-P/LP variants are increasingly identified in early breast cancer oncology practice as a result of greater acceptance of the influence of gBRCA-P/LP variant status on treatment choices.³⁶ The OlympiA Trial provides evidence that germline BRCA1 and BRCA2 sequencing is an important biomarker for the selection of systemic therapy in early breast cancer.

### References

1. Breast Cancer Association C, Dorling L, Carvalho S, et al. Breast Cancer Risk Genes—Association Analysis in More than 113,000 Women. N Engl J Med 2021;384:428-439.
2. Hu C, Hart SN, Gnanaolivu R, et al. A Population-Based Study of Genes Previously Implicated in Breast Cancer. N Engl J Med 2021;384:440-451.
3. Kuchenbaecker KB, Hopper JL, Barnes DR, et al. Risks of Breast, Ovarian, and Contralateral Breast Cancer for BRCA1 and BRCA2 Mutation Carriers. JAMA 2017;317:2402-2416.
4. Mavaddat N, Barrowdale D, Andrulis IL, et al. Pathology of breast and ovarian cancers among BRCA1 and BRCA2 mutation carriers: results from the Consortium of Investigators of Modifiers of BRCA1/2 (CIMBA). Cancer Epidemiol Biomarkers Prev 2012;21:134-47.
5. Atchley DP, Albarracin CT, Lopez A, et al. Clinical and pathologic characteristics of patients with BRCA-positive and BRCA-negative breast cancer. J Clin Oncol 2008;26:4282-8.
6. Lakhani SR, Van De Vijver MJ, Jacquemier J, et al. The pathology of familial breast cancer: predictive value of immunohistochemical markers estrogen receptor, progesterone receptor, HER-2, and p53 in patients with mutations in BRCA1 and BRCA2. J Clin Oncol 2002;20:2310-8.
7. NCCN Genetic/Familial High-Risk Assessment: Breast and Ovarian Version 1.2020. National Comprehensive Cancer Network, April 2021. (https://www.nccn.org/store/login/login.aspx?ReturnURL=https://www.nccn.org/professio nals/physician gls/pdf/genetics_bop.pdf).
8. Tutt A, Ashworth A. The relationship between the roles of BRCA genes in DNA repair and cancer predisposition. Trends Mol Med 2002;8:571-6.
9. Davies H, Glodzik D, Morganella S, et al. HRDetect is a predictor of BRCA1 and BRCA2 deficiency based on mutational signatures. Nat Med 2017;23:517-525.
10. Staaf J, Glodzik D, Bosch A, et al. Whole-genome sequencing of triple-negative breast cancers in a population-based clinical study. Nat Med 2019;25:1526-1533.
11. Tutt AN, Lord CJ, McCabe N, et al. Exploiting the DNA repair defect in BRCA mutant cells in the design of new therapeutic strategies for cancer. Cold Spring Harb Symp Quant Biol 2005;70:139-48.
12. Farmer H, McCabe N, Lord CJ, et al. Targeting the DNA repair defect in BRCA mutant cells as a therapeutic strategy. Nature 2005;434:917-21.
13. Bryant HE, Schultz N, Thomas HD, et al. Specific killing of BRCA2-deficient tumours with inhibitors of poly(ADP-ribose) polymerase. Nature 2005;434:913-7.
14. Fong PC, Boss DS, Yap TA, et al. Inhibition of poly(ADP-ribose) polymerase in tumors from BRCA mutation carriers. N Engl J Med 2009;361:123-34.
15. Tutt A, Robson M, Garber JE, et al. Oral poly(ADP-ribose) polymerase inhibitor olaparib in patients with BRCA1 or BRCA2 mutations and advanced breast cancer: a proof-of-concept trial. Lancet 2010;376:235-44.
16. Audeh MW, Carmichael J, Penson RT, et al. Oral poly(ADP-ribose) polymerase inhibitor olaparib in patients with BRCA1 or BRCA2 mutations and recurrent ovarian cancer: a proof-of-concept trial. Lancet 2010;376:245-51.
17. Kaufman B, Shapira-Frommer R, Schmutzler RK, et al. Olaparib monotherapy in patients with advanced cancer and a germline BRCA1/2 mutation. J Clin Oncol 2015;33:244-50.
18. Shah PD, Patil S, Dickler MN, Offit K, Hudis CA, Robson ME. Twenty-one-gene recurrence score assay in BRCA-associated versus sporadic breast cancers: Differences based on germline mutation status. Cancer 2016;122:1178-84.
19. Tryggvadottir L, Olafsdottir EJ, Olafsdottir GH, et al. Tumour diploidy and survival in breast cancer patients with BRCA2 mutations. Breast Cancer Res Treat 2013;140:375-84.
20. Mittendorf EA, Jeruss JS, Tucker SL, et al. Validation of a novel staging system for disease-specific survival in patients with breast cancer treated with neoadjuvant chemotherapy. J Clin Oncol 2011;29:1956-62.
21. Hudis CA, Barlow WE, Costantino JP, et al. Proposal for standardized definitions for efficacy end points in adjuvant breast cancer trials: the STEEP system. J Clin Oncol 2007;25:2127-32.
22. Stone A. The application of bespoke spending functions in group-sequential designs and the effect of delayed treatment switching in survival trials. Pharm Stat 2010;9:151-61.
23. Cuzick J. Forest plots and the interpretation of subgroups. Lancet 2005;365:1308.
24. Robson M, Im SA, Senkus E, et al. Olaparib for Metastatic Breast Cancer in Patients with a Germline BRCA Mutation. N Engl J Med 2017;377:523-533.
25. Litton JK, Rugo HS, Ettl J, et al. Talazoparib in Patients with Advanced Breast Cancer and a Germline BRCA Mutation. N Engl J Med 2018;379:753-763.
26. Fasching PA, Link T, Hauke J, et al. Neoadjuvant paclitaxel/olaparib in comparison to paclitaxel/carboplatinum in patients with HER2-negative breast cancer and homologous recombination deficiency (GeparOLA study). Ann Oncol 2021;32:49-57.
27. Burstein HJ, Curigliano G, Loibl S, et al. Estimating the benefits of therapy for early-stage breast cancer: the St. Gallen International Consensus Guidelines for the primary therapy of early breast cancer 2019. Ann Oncol 2019;30:1541-1557.
28. NCCN Clinical Practice Guidelines in Oncology-Breast Cancer Version 5.2020. National Comprehensive Cancer Network, April 2021. (https://www2.tri-kobe.org/nccn/guideline/breast/english/breast.pdf).
29. Tutt A, Tovey H, Cheang MCU, et al. Carboplatin in BRCA1/2-mutated and triple-negative breast cancer BRCAness subgroups: the TNT Trial. Nat Med 2018;24:628-637.
30. Zhang J, Lin Y, Sun XJ, et al. Biomarker assessment of the CBCSG006 trial: a randomized phase III trial of cisplatin plus gemcitabine compared with paclitaxel plus gemcitabine as first-line therapy for patients with metastatic triple-negative breast cancer. Ann Oncol 2018;29:1741-1747.
31. Murai J, Huang SY, Das BB, et al. Trapping of PARP1 and PARP2 by Clinical PARP Inhibitors. Cancer Res 2012;72:5588-99.
32. Morice PM, Leary A, Dolladille C, et al. Myelodysplastic syndrome and acute myeloid leukaemia in patients treated with PARP inhibitors: a safety meta-analysis of randomised controlled trials and a retrospective study of the WHO pharmacovigilance database. Lancet Haematol 2021;8:e122-e134.
33. Pohl-Rescigno E, Hauke J, Loibl S, et al. Association of Germline Variant Status With Therapy Response in High-risk Early-Stage Breast Cancer: A Secondary Analysis of the GeparOcto Randomized Clinical Trial. JAMA Oncol 2020;6:744-748.
34. Masuda N, Lee SJ, Ohtani S, et al. Adjuvant Capecitabine for Breast Cancer after Preoperative Chemotherapy. N Engl J Med 2017;376:2147-2159.
35. Robson ME, Tung N, Conte P, et al. OlympiAD final overall survival and tolerability results: Olaparib versus chemotherapy treatment of physician's choice in patients with a germline BRCA mutation and HER2-negative metastatic breast cancer. Ann Oncol 2019;30:558-566.
36. Tutt A, Ashworth A. Can genetic testing guide treatment in breast cancer? Eur J Cancer 2008;44:2774-80.

TABLE 1
Demographic and Baseline Disease Characteristics of the Patients.*

| | Olaparib Group | | Placebo Group | |
|---|---|---|---|---|
| Characteristic | (n = 921) | | (n = 915) | |
| Age, years - median (interquartile range) | 42 | (36-49) | 43 | (36-50) |
| BRCA gene - no. (%)† | | | | |
| BRCA1 | 657 | (71.3) | 670 | (73.2) |
| BRCA2 | 261 | (28.3) | 239 | (26.1) |
| BRCA1 and BRCA2 | 2 | (0.2) | 5 | (0.5) |
| Missing | 1 | (0.1) | 1 | (0.1) |
| Prior adjuvant/neoadjuvant | | | | |
| chemotherapy - no. (%) | | | | |
| Adjuvant | 461 | (50.1) | 455 | (49.7) |
| Neoadjuvant | 460 | (49.9) | 460 | (50.3) |
| Anthracycline and taxane regimen | 871 | (94.6) | 849 | (92.8) |
| Anthracycline regimen (without taxane) | 7 | (0.8) | 13 | (1.4) |
| Taxane regimen (without anthracycline) | 43 | (4.7) | 52 | (5.7) |
| Regimen not reported. | 0 | (0.0) | 1 | (0.1) |
| Less than six cycles of neoadjuvant or | 7 | (0.8) | 15 | (1.6) |
| adjuvant chemotherapy | | | | |
| Neoadjuvant or adjuvant platinum-based | | | | |
| therapy - no. (%) | | | | |
| No | 674 | (73.2) | 676 | (73.9) |
| Yes | 247 | (26.8) | 239 | (26.1) |
| Concurrent hormone therapy | 146/168 | (86.9) | 142/157 | (90.4) |
| (hormone receptor-positive only) - no. (%) | | | | |
| Hormone receptor status - no. (%)‡ | | | | |
| Hormone receptor-positive/HER2-negative§ | 168 | (18.2) | 157 | (17.2) |
| Triple-negative breast cancer¶ | 751 | (81.5) | 758 | (82.8) |
| Menopausal status (females only) - no. (%) | | | | |
| Premenopausal | 572/919 | (62.2) | 553/911 | (60.7) |
| Postmenopausal | 347/919 | (37.8) | 358/911 | (39.3) |
| Primary breast cancer surgery - no. (%) | | | | |
| Mastectomy | 698 | (75.8) | 673 | (73.6) |
| Conservative surgery only | 223 | (24.2) | 240 | (26.2) |
| Missing | 0 | (0.0) | 2 | (0.2) |

*Further patient demographics and baseline disease characteristics are shown in Table S6 in the Supplementary Appendix.
HER2 denotes human epidermal growth factor receptor 2;
†For a detailed description of local and central Myriad BRCA testing in patients enrolled on OlympiA please see FIG. S2 in the Supplementary Appendix. Variant interpretation by Myriad Genetics (BRCAnalysis) (n = 1561) and BGI Genomics (n = 247) is performed using multiple established databases (e.g., ClinVar, ClinGen, ENIGMA) and published and internal functional and clinical data, compliant with ACMG published guidelines. The 24 P/LP variants from local labs without central Myriad confirmation were confirmed by the OlympiA Genetics Advisory Committeeusing published databases as above. Discordant data are enumerated in the FIG. Table S2 in the Supplementary Appendix lists P/LP (D/SD) BRCA1 and BRCA 2 variants occurring in more than 1 patient.
‡Defined by local test results.
§The original protocol activated in 2014 was developed for HER2-negative patients but included only patients with triple-negative breast cancer following regulatory review. When hormone receptor-positive recurrence risk and olaparib and endocrine therapy combination safety rationale was accepted by regulators, the protocol was amended in 2015 to include patients with high-risk hormone receptor-positive disease and increase the sample size to the current number of 1800 patients (see Protocol History on www.nejm.org). The first patient with hormone receptor-positive disease was enrolled in December 2015.
¶Triple negative breast cancer was defined in eligibility criteria as: ER and PgR negative defined as IHC nuclear staining <1%. AND HER2 negative (not eligible for anti-HER2 therapy) defined as: IHC 0, 1+ without ISH OR IHC 2+ and ISH non-amplified with ratio less than 2.0 and if reported, average HER2 copy number <4 signals/cells OR ISH non-amplified with ratio less than 2.0 and if reported, average HER2 copy number <4 signals/cells (without IHC)
Two patients are excluded from the summary of the triple-negative breast cancer subset because they do not have confirmed HER2-negative status.

TABLE 2
Adverse events of any grade with an incidence of at least 10% in either treatment arm in the safety analysis set.

| | Olaparib (n = 911) | | | | | | | | Placebo (n = 904) | | | | | | |
|---|---|---|---|---|---|---|---|---|---|---|---|---|---|---|---|
| Adverse Event - no. (%) | Any Grade | | Grade 1 | | Grade 2 | | Grade ≥3* | | Any Grade | | Grade 1 | | Grade 2 | | Grade ≥3* |
| Nausea | 518 | (56.9) | 390 | (42.8) | 121 | (13.3) | 7 | (0.8) | 211 | (23.3) | 185 | (20.5) | 26 | (2.9) | 0 (0.0) |
| Fatigue | 365 | (40.1) | 240 | (26.3) | 109 | (12.0) | 16 | (1.8) | 245 | (27.1) | 188 | (20.8) | 53 | (5.9) | 4 (0.4) |
| Anemia | 214 | (23.5) | 68 | (7.5) | 67 | (7.4) | 79 | (8.7) | 35 | (3.9) | 19 | (2.1) | 13 | (1.4) | 3 (0.3) |
| Vomiting | 206 | (22.6) | 160 | (17.6) | 40 | (4.4) | 6 | (0.7) | 74 | (8.2) | 64 | (7.1) | 10 | (1.1) | 0 (0.0) |
| Headache | 180 | (19.8) | 145 | (15.9) | 33 | (3.6) | 2 | (0.2) | 152 | (16.8) | 120 | (13.3) | 31 | (3.4) | 1 (0.1) |
| Diarrhea | 160 | (17.6) | 125 | (13.7) | 32 | (3.5) | 3 | (0.3) | 124 | (13.7) | 96 | (10.6) | 25 | (2.8) | 3 (0.3) |
| Neutrophil | 146 | (16.0) | 36 | (4.0) | 66 | (7.2) | 44 | (4.8) | 59 | (6.5) | 17 | (1.9) | 35 | (3.9) | 7 (0.8) |
| count decreased | | | | | | | | | | | | | | | |
| White blood cell | 143 | (15.7) | 41 | (4.5) | 75 | (8.2) | 27 | (3.0) | 52 | (5.8) | 27 | (3.0) | 22 | (2.4) | 3 (0.3) |
| count decreased | | | | | | | | | | | | | | | |
| Decreased appetite | 119 | (13.1) | 101 | (11.1) | 16 | (1.8) | 2 | (0.2) | 53 | (5.9) | 45 | (5.0) | 8 | (0.9) | 0 (0.0) |
| Dysgeusia | 107 | (11.7) | 101 | (11.1) | 6 | (0.7) | 0 | (0.0) | 38 | (4.2) | 36 | (4.0) | 2 | (0.2) | 0 (0.0) |
| Dizziness | 104 | (11.4) | 91 | (10.0) | 12 | (1.3) | 1 | (0.1) | 67 | (7.4) | 61 | (6.7) | 5 | (0.6) | 1 (0.1) |
| Arthralgia | 84 | (9.2) | 60 | (6.6) | 22 | (2.4) | 2 | (0.2) | 107 | (11.8) | 85 | (9.4) | 20 | (2.2) | 2 (0.2) |

*All listed adverse events are grade 3 except for 10 grade 4 events in the olaparib arm: (neutrophil count decreased, n = 5; anemia, n = 4; fatigue, n = 1).

TABLE 3
Summary of adverse events in the safety analysis set.*

| Adverse Event - | Olaparib | | Placebo | |
|---|---|---|---|---|
| no. of patients (%) | (N = 911) | | (N = 904) | |
| Any adverse event | 835 | (91.7) | 753 | (83.3) |
| Serious adverse event | 79 | (8.7) | 76 | (8.4) |
| Adverse event of special | 30 | (3.3) | 46 | (5.1) |
| interest† | | | | |
| MDS/AML | 2 | (0.2) | 3 | (0.3) |
| Pneumonitis | 9 | (1.0) | 11 | (1.2) |
| New primary malignancy | 20 | (2.2) | 32 | (3.5) |
| Grade ≥3 adverse event | 221 | (24.3) | 102 | (11.3) |
| Grade 4 adverse event‡ | 17 | (1.9) | 4 | (0.4) |
| Adverse event leading to | 90 | (9.9) | 38 | (4.2) |
| permanent discontinuation | | | | |
| of treatment§ | | | | |
| Adverse event leading | 1 | (0.1) | 2 | (0.2) |
| to death¶ | | | | |

*Includes adverse events with an onset date on or after the first dose date and up to and including 30 days following date of last dose of study medication. AML denotes acute myeloid leukemia; MDS myelodysplastic syndrome.
†Includes adverse events of special interest with onset at any date after first dose of treatment: MDS/AML(olaparib, n = 2; placebo, n = 3); pneumonitis (olaparib, n = 7; placebo, n = 8); radiation pneumonitis (olaparib, n = 2; placebo, n = 3); new primary invasive breast cancer (olaparib, n = 6; placebo, n = 7); new primary breast cancer and new serous tubular intraepithelial carcinoma (placebo, n = 1); new primary breast cancer and new lung cancer (olaparib, n = 1); new primary ductal carcinoma in situ (olaparib, n = 3, placebo, n = 4); new primary ovarian malignancy (olaparib, n = 1; placebo, n = 4, one of which is a possible recurrence of ovarian cancer >5 years before randomization); new primary fallopian tube cancer (olaparib, n = 1; placebo, n = 4); new primary lung cancer (olaparib, n = 1; placebo, n = 2) malignant melanoma (olaparib, n = 1; placebo, n = 3); non-melanoma skin malignancy (olaparib, n = 3; placebo, n = 2); endometrial adenocarcinoma (olaparib, n = 1; placebo, n = 1); colorectal cancer, meningioma (olaparib, n = 1 for both); cervix carcinoma, pancreatic carcinoma, rectal carcinoma, transitional cell carcinoma (placebo, n = 1 for each). One patient in the olaparib arm had both pneumonitis and a non-melanoma skin malignancy and is counted in both categories.
‡Eighteen grade 4 AEs were reported in 17 patients assigned to olaparib; one patient had both grade 4 anemia and neutrophil count decreased. Grade 4 AEs include neutrophil count decreased (olaparib, n = 5); anemia (olaparib, n = 4); lymphocyte count decreased (olaparib, n = 3); AML, bipolar disorder, fatigue, febrile neutropenia, hepatic function abnormal and suicide attempt (olaparib, n = 1 for each); depression (placebo, n = 2); aspartate aminotransferase increased, cholecystitis acute (placebo, n = 1 for each).
§The most common adverse events, occurring in ≥1% of the patients, leading to discontinuation of study drug in the olaparib group were: nausea (2.0%), anemia (1.8%), fatigue (1.3%), and neutrophil count decreased (1%); there were no adverse events that occurred in ≥1% of patients leading to discontinuation of study drug in the placebo group.
¶Adverse events leading to death are cardiac arrest (olaparib, n = 1), AML (placebo, n = 1), andovarian cancer (placebo, n = 1).

### Supplementary Appendix

This supplementary appendix has been provided to give the reader additional information about the described methods of treating cancer.

### 3. SUPPLEMENTARY METHODS

### 3.1 Dual Platform Model Used to Conduct the Olympia Trial

This trial was conducted as a partnership between academia, non-profit organisations, government agencies, participating hospitals and industry. The Breast International Group (BIG), Frontier Science and Technology Research Foundation (and its Affiliate, Frontier Science (Scotland) Ltd), the National Cancer Institute, NRG Oncology and AstraZeneca have all played key roles. The guiding principles for the conduct of the study are those of BIG and NRG/NCI. Data is collected, reviewed and analysed following the Standard Operating Procedures of Frontier Science (non-profit organisation) and NRG/NCI. All of these organisations have representation on the trial Steering Committee along with representatives of the geographic areas involved in the trial and consumer representatives. A detailed Publication Policy governs all publications using trial data and decisions to publish come from the Steering Committee, not from any individual or individual organization.

Two protocols, identical in terms of study objectives and scientific content differing only in logistical and regulatory content appropriate for the country(ies) they covered (eg. drug distribution, mechanisms for SAE reporting during the study, etc), are employed in the study.

The protocol under AZ sponsorship covers all patients recruited from non-US sites and the protocol under NRG sponsorship covers patients within the US. The protocols were developed as a collaboration between the partners described above.

The trial used a single randomization system hosted by Frontier Science (FS) and is reported as one study. Randomization was done using a permuted block algorithm with block-size 4. The randomization system has a built-in random number generator to start the allocations, and blocks are generated randomly as they are required, so there are no random lists generated ahead of time. Non-US sites used the FS front end to get into the randomization system. US sites used the NCI OPEN system which collected pre-randomization information and then connected to the FS system to complete randomization. All patients, treating physicians, and study personnel were blinded to treatment allocation with exception of the Independent Statistical Center, which was provided with treatment codes by the randomization system administrator in order to prepare reports for the Independent Data Monitoring Committee (IDMC).

The collection of the patient data is done using two instances of Rave EDC system (one for the US patients, maintained by NRG, and one for all other patients outside of the US, maintained by FS)). FS and NRG collaborated on the design of the two databases and the respective eCRFs to ensure as much consistency as possible in the data collection. Some differences have been necessary due to differences in company and/or regional data collection standards and these differences are all documented in consistency documentation maintained by AZ. Quality control of the data is done by Frontier Science and NRG for the respective Rave instances. The data from both databases are routinely combined into a single consolidated database at regular intervals. All statistical analyses as well as reports for periodic review by the IDMC have been conducted and reported from the single consolidated database, built, maintained and held by Frontier Science. The Sponsors (NRG/NCI and AstraZeneca) had no access to this database during the conduct of the trial. Subsets of blinded data were provided for specific purposes as required, e.g. DSUR reporting data to AZ and a subset of PRO data to NRG to allow them to test analysis programs.

### 3.2 Full Eligibility Criteria

1. Provision of informed consent prior to any study specific procedures
2. Female or male patients must be ≥18 years of age
3A. For patients who underwent initial surgery and received adjuvant chemotherapy
   TNBC patients must have been axillary node-positive (≥pN1, any tumour size) or axillary node-negative (pN0) with invasive primary tumour pathological size>2 cm (≥pT2)
   □ER and/or PgR positive/HER 2 negative patients must have had ≥4 pathologically confirmed positive lymph nodes
3B. For patients who underwent neoadjuvant chemotherapy followed by surgery
   TNBC patients must have residual invasive breast cancer in the breast and/or resected lymph nodes (non pCR)
   ER and/or PgR positive/HER 2 negative patients must have residual invasive cancer in the breast and/or the resected lymph nodes (non pCR) AND a CPS&EG score≥3. Instructions how to calculate CPS&EG score (Mittendorf et al 2011; Jeruss et al 2008) are provided in Appendix 4.
4. Histologically confirmed non-metastatic primary invasive adenocarcinoma of the breast that is one of the two following phenotypes:
a) TNBC defined as:
   ER and PgR negative defined as IHC nuclear staining<1%.
AND
   HER2 negative (not eligible for anti-HER2 therapy) defined as:
      IHC 0, 1+ without ISH OR
      IHC 2+ and ISH non-amplified with ratio less than 2.0 and if reported, average HER2 copy number<4 signals/cells OR
      ISH non-amplified with ratio less than 2.0 and if reported, average HER2 copy number<4 signals/cells (without IHC)
b) ER and/or PgR positive, HER2 negative breast cancer defined as:
   ER and/or PgR positive defined as IHC nuclear staining≥1%.
AND
   HER2 negative (not eligible for anti-HER2 therapy) defined as:
      IHC 0, 1+without ISH OR
      IHC 2+and ISH non-amplified with ratio less than 2.0 and if reported, average HER2 copy number<4 signals/cells OR
      ISH non-amplified with ratio less than 2.0 and if reported, average HER2 copy number<4 signals/cells (without IHC)
Patients with multifocal or multicentric invasive disease are eligible as long as all the lesions for which HER2 characterization is available are HER2 negative.
Patients with synchronous bilateral invasive disease are eligible as long as all the lesions assessed for HER2 on both sides are negative.
In both the above cases the lesion considered at highest risk for recurrence based on the investigator's discretion will be used for eligibility determination.
5. Documented germline mutation in BRCA1 or BRCA2 that is predicted to be deleterious or suspected
   deleterious (known or predicted to be detrimental/lead to loss of function). Local gBRCA testing results, if available, will be used for establishing eligibility. If local gBRCA testing results are not available, central testing will be provided for those patients who otherwise appear to be eligible (see Section 6.2.1).
6A. Completed adequate breast surgery defined as:
   The inked margins of breast conservation surgery or mastectomy must be histologically free of invasive breast cancer and ductal carcinoma in situ with the exception of the posterior margin if this margin is the pectoralis major fascia or the anterior margin if this is the dermis. Patients with resection margins positive for lobular carcinoma in situ are eligible.
   Patients with breast conservation must have adjuvant radiotherapy. Patients having mastectomy may have adjuvant radiotherapy according to local policy and/or international guidelines.
6B. Completed adequate axilla surgery defined as:
Adjuvant Chemotherapy Patients:
   Sentinel lymph node biopsy alone if negative or if lymph node(s) only contain micrometastases ($2.0 mm) OR
   Positive sentinel lymph node biopsy followed by axillary nodal dissection or radiotherapy as per local guidelines OR
   Axillary dissection
Neoadjuvant Chemotherapy Patients:
   Sentinel lymph node biopsy performed before neoadjuvant chemotherapy:
      If negative or if lymph node(s) only contain micrometastases ($2.0 mm) additional axillary surgery is not required
      If positive, axillary node dissection or axillary nodal radiotherapy should follow completion of neoadjuvant chemotherapy
   Sentinel lymph node biopsy performed after neoadjuvant chemotherapy:
      If negative, additional axillary surgery not mandated
      If positive (micrometastases are regarded as positive), additional axillary surgery is required unless the patient is enrolled in a Phase III multicenter clinical trial proposing radiotherapy as alternative treatment of the axilla. The trial must be pre-approved by the OlympiA Executive Committee
   Axillary dissection
7. Completed at least 6 cycles of neoadjuvant or adjuvant chemotherapy containing anthracyclines, taxanes or the combination of both. Prior platinum as potentially curative treatment for prior cancer (e.g. ovarian) or as adjuvant or neoadjuvant treatment for breast cancer is allowed. (For neoadjuvant patients all chemotherapy should be delivered prior to surgery. No further cycles of chemotherapy post surgery are allowed.)
8. Patients must have adequate organ and bone marrow function measured within 28 days prior to randomisation with no blood transfusions (packed red blood cells and/or platelet transfusions) in the past 28 days prior to testing for organ and bone marrow function as defined below:
   Haemoglobin≥10.0 g/dL
   Absolute neutrophil count (ANC)≥1.5×109/L
   Platelet count≥100×109/L
   Total Bilirubin≤ULN (institutional upper limit of normal) except elevated total bilirubin <1.5×ULN due to Gilbert's disease or similar syndrome involving slow conjugation of bilirubin
   AST (SGOT)/ALT (SGPT)≤2.5×ULN
   ALP≤2.5×ULN

To rule out metastatic breast cancer, patients with screening ALT/AST or ALP above institutional upper limit of normal should have liver ultrasound, CT or MRI at any time point between diagnosis of current breast cancer and randomisation.

Screening bone scan is required if ALP and/or corrected calcium level are above the institutional upper limit. (Note: PET CT scan may be used as an alternative imaging technique).
9. Serum or plasma creatinine≤1.5×ULN
10. ECOG performance status 0-1
11A. Women who are not postmenopausal or have not undergone hysterectomy must have documented negative pregnancy test within 28 days prior to randomisation:
Postmenopausal is defined as:
   Age≥60 years
   Age<60 years and amenorrheic for 1 year or more in the absence of chemotherapy and/or hormonal treatment
   Follicle stimulating hormone (FSH) and plasma estradiol levels in the postmenopausal range for women under 60 years
   Radiation-induced oophorectomy with last menses>1 year ago
   Bilateral oophorectomy
11B. Women of child bearing potential and their partners, who are sexually active, must agree to the use of two highly effective forms of contraception in combination. This should be started from the signing of the informed consent and continue, throughout the period of taking study treatment and for at least 1 month after last dose of study drug, or they must totally/truly abstain from any form of sexual intercourse. Male patients must use a condom during treatment and for 3 months after last dose of study drug when having sexual intercourse with a pregnant woman or with a woman of childbearing potential. Female partners of male patients should also use a highly effective form of contraception (see Appendix E for acceptable methods) if they are of childbearing potential.
12. Patient is willing and able to comply with the protocol for the duration of the study including undergoing treatment and scheduled visits and examinations
13. Formalin fixed, paraffin embedded (FFPE) tumour sample from the primary tumour, mandatory*.

*NOTE: For adjuvant patients, this refers to the surgical specimen; for neoadjuvant patients, both the pre-treatment core biopsy and the surgical specimen with residualdisease are requested but only one is mandatory. If the surgery tumour blocks are available, but cannot be submitted, sites may submit a portion of invasive tumour from the original block, either by taking at least one core of at least 3 mm in diameter, or by splitting the original block in two parts, and re-embedding one in a new block for central submission. If blocks containing pre-neoadjuvant treatment core biopsies are available but cannot be submitted, sections mounted on glass slides prepared from the block can be provided. If tumour sample can't be provided as requested above or if it's not available, approval by Study Team for patient's entry into the trial is required.
14. Patient should be randomised in the trial ideally within a maximum of 8 weeks of completion of their last treatment (surgery, chemotherapy or radiotherapy), but in no case longer than 12 weeks.

### 3.3 CALCULATION FOR THE CPS&EG STAGING SYSTEM

The CPS&EG score is a staging system for disease specific survival in patients with breast cancer treated with neoadjuvant chemotherapy.1 This incorporates pretreatment clinical stage, estrogen receptor status, nuclear grade and post-neoadjuvant chemotherapy pathological stage.

Calculation instructions:

Add the points for Clinical Stage+Pathologic Stage+ER status+Nuclear grade to derive a sum (CPS&EG score) between 0 and 6.

| Stage/feature | | Points |
|---|---|---|
| Clinical Stage | 0 | 0 |
| (AJCC staging [1]) | IIA | 0 |
| | IIB | 1 |
| | IIIA | 1 |
| | IIIB | 2 |
| | IIIC | 2 |
| Pathologic Stage | 0 | 0 |
| (AJCC staging [1]) | I | 0 |
| | IIA | 1 |
| | IIB | 1 |
| | IIIA | 1 |
| | IIIB | 1 |
| | IIIC | 2 |
| Receptor status | ER negative [2] | 1 |
| | Nuclear grade 3 | 1 |
| Nuclear grade [3] | | |

[1] AJCC: American Joint Committee on Cancer (https://cancerstaging.org/Pages/default.aspx).
[2] ER: Estrogen receptor; definitions for ER negativity see eligibility criteria in the protocol Section 4.1.4.a.
[3] In the unlikely situation nuclear grade cannot be determined, regular histologic grade should be used; if only Nottingham overall grade is reported, the Nottingham overall grade must be 9 to be scored as 1 point in the CPS&EG score (http://pathology.jhu.edu/breast/grade.php).

### 3.4 POOLING STRATEGY FOR STRATIFICATION FACTORS

The primary stratified log-rank test of IDFS will be based on the stratification factors determined from the following pooling strategy.

In the event that there are fewer than 5 IDFS events per treatment arm within any individual stratum (initially starting with 16 strata; 16=2×2×2×2 including treatment group), one stratification factor will be removed at a time until there are at least 5 IDFS events within each individual stratum in the following order:
1. Prior platinum use for breast cancer (yes/no)
2. Prior chemotherapy (neo-adjuvant vs. adjuvant)
3. Hormone receptor status (ER and/or PgR positive/HER2 negative vs. TNBC)

Result: When all three factors were included, there were strata with fewer than 5 IDFS events per treatment arm. Hence, prior platinum was removed as a stratification factor. When the remaining two factors were included, there were strata with fewer than 5 IDFS events per treatment arm. Hence, prior chemotherapy was removed as a stratification factor. Therefore, the primary stratified Cox proportional hazards model and the stratified log-rank test of IDFS were based on the stratification factor of hormone receptor status only.

### 3.5 SENSITIVITY ANALYSES

The protocol specified that seven (7) sensitivity analyses were to be performed if specific criteria were met. In this section we describe the sensitivity analyses, and, for those that met the criteria for conducting the sensitivity analysis, results are presented in tables within this Supplementary Appendix.

### 1: Confirmed (Central Myriad Test) Germline BRCA1 and BRCA2 Deleterious/Suspected Deleterious Variant

The protocol specified that, If applicable, an analysis would be performed for IDFS based on all randomised patients confirmed to have BRCA1 or BRCA2 germline deleterious/suspected deleterious variant (gBRCA-D/SD-variant) by the central Myriad test. This analysis is only required if the analysis population differs from the primary ITT population (i.e. only required if any of the randomised patients are not confirmed to have gBRCA-D/SD-variant by the central Myriad test).

1539 patients had a Myriad confirmed gBRCA D/SD variant (see Table S2 in this Supplementary Appendix).

Results: The results of this analysis are presented within Table S9 in this Supplementary Appendix.

### 2: Mis-stratification in the Randomisation System

Any patients mis-stratified in the randomisation system (i.e. incorrect details are entered at the time of randomisation) were included in the primary stratified analysis based on the information from the randomisation system. Cross-tabulations of stratification factors from the randomisation system and the correct baseline data from the eCRF were performed. If >5% of randomised patients are incorrectly stratified (i.e. randomisation system data does not match baseline data confirmed in the eCRF) then a sensitivity analysis would be performed for IDFS using the same model as described above but using the eCRF information instead of the randomisation system information. [Note: For all patients, the characteristics reported in the eCRF were used to determine subgroups for the subgroup analyses, while the randomisation system information was used to stratify the logrank and Cox model analyses.]

In accordance with the pooling strategy only hormone receptor status was fitted as a stratification factor. Of the 1836 in the ITT population, 32 (1.7%) had discordant hormone receptor status between what was reported in the randomisation system and what was reported on the eCRF.

Results: Because the 5% threshold was not met, this sensitivity analysis was not performed.

### 3: Central Pathology Review

The protocol specified that if the results of ER and PgR status from the local and central labs differ in >5% of randomised patients, then a sensitivity analysis would be performed for IDFS using the same model as described above, but using the central lab result to determine the HR status stratification factor and compared with the primary analysis result.

Of the 1452 patients that have both a central and a local hormone receptor status, 147 (10%) have discordant results (Table S5 in this Supplementary Appendix). 247 patients did not have material available for central pathology review because of regulatory requirements by authorities in China. Central receptor status review results excluding patients from China are shown in Table S4 in this Supplementary Appendix.

Results: Because the 5% threshold for discordance between local and central hormone receptor status was met, this sensitivity analysis was performed. The results of this analysis are presented in Table S9 in this Supplementary Appendix.

### 4: Important Protocol Deviations (IPDS)

Important protocol deviations (IPD)s are a concise list of pre-defined protocol deviations which have a very high likelihood of influencing the primary efficacy and/or the secondary safety results. The protocol stated that a ‘deviation bias’ sensitivity analysis may be performed excluding patients with IPD's that may affect the efficacy of the trial therapy. This sensitivity analysis would be performed excluding patients with IPD's that may affect the efficacy of the trial therapy if >10% of patients in either treatment group did not have the intended disease or indication or did not receive any randomised therapy.

Of the 1836 patients in the ITT population, 30 (1.6%) did not have intended disease or indication, or did not receive any randomised treatment (see Table S18 in this Supplementary Appendix).

Results: Because the 10% threshold for IPDs was not met, this sensitivity analysis was not performed.

5. Unadjusted analysis

The protocol stated that an unadjusted (unstratified Cox model) analysis would be performed as a sensitivity analysis and compared with the primary results.

Results: This unstratified Cox model analysis was performed. The results of this analysis are presented in Table S9 in this Supplementary Appendix.

6. Assumption of proportional hazards

The protocol stated that the assumption of proportional hazards underlying the log-rank test and the Cox model used for the primary analysis would be assessed. Proportionality will be assessed using two approaches, firstly by inspecting plots of complementary log-log (time) versus log (time) and secondly by formally testing using the Grambsch-Therneau test (G-T) based on scaled Schoenfeld residuals from a Cox model including treatment group as a factor. If the G-T test is significant (p<0.05), and proportionality is rejected, Restricted Mean Survival Time (RMST) methods would be used to estimate and test the treatment difference while allowing for non-proportional hazards.

Results: The G-T tests reached the p<0.05 threshold. This indicates that proportional hazards cannot be assumed. a rejection of the null hypothesis of proportional hazards. The p-value for the G-T test with identity transformation of time was p=0.02, and the p-value for the G-T test with rank transformation of time was p=0.02 (see Table S9 in this Supplementary Appendix).

Because the null hypothesis of proportionality was rejected, as specified in the Statistical Analysis Plan, a sensitivity analysis was performed based on the restricted mean survival time (RMST) method, restricting the calculation of RMST to within the first 4.1 years (49 months) of follow-up. The restriction time was defined as the minimum of the maximum of the longest IDFS event time between the two treatment groups. Under non-proportional hazards, the estimated hazard ratio can be interpreted as an average hazard ratio over the observed follow-up period. This hazard ratio may under and overestimate the hazard during different periods of the follow-up. The results of the RMST analysis reach the same conclusion as the main analysis of IDFS, that there is a treatment benefit for the olaparib group. The results of the RMST analysis is presented in Table S9 in this Supplementary Appendix.

### 7. Interval Censored Cox Regression

The protocol stated that an interval censored analysis would be performed as a sensitivity analysis and compared with the primary results. Patients whose visit schedule has not been according to the protocol are fitted in the Cox model using interval censoring,
For patients experiencing an event, and without follow-up according to the protocol (defined as over 18 months between the event and the last visit), the interval from the last date at which the subject was known to be IDFS free to the date of recurrence or death, will be used.
For patients that were previously censored, or had an event and were seen according to the protocol defined visit schedule, the lower limit of the interval will be set to the censoring/event date, while the upper limit will be set to missing.

Results: No patients met the criteria to initiate this sensitivity analysis.

### 4. SUPPLEMENTARY FIGURES

### FIGURE S5: EORTC QLQ-C30 GHQ SCORE

The primary objective of the planned Patient Reported Outcomes (PRO) substudy is to determine the effect of olaparib on patient-reported fatigue at 6 and 12 months after randomization as measured by FACIT-Fatigue. Evaluating the effect of olaparib on health-related quality of life over the first two years from randomization is one of the secondary objectives of the PRO substudy. This is measured by the 2-item General Health Status/Quality of Life (GHQ) scale of the EORTC QLQ-C30 questionnaire. Data for the protocol planned analysis of PROs in Olympia are immature, with only half the study sample with data available at 2 years, and so are not reported at this time. In addition, the PRO data analysis plan stratifies the study sample and considers separate analyses for those who received neoadjuvant or adjuvant chemotherapy prior to trial randomization. Here we show plots of mean EORTC QLQ-C-30 GHQ score by treatment assignment for patients who received neoadjuvant therapy and adjuvant chemotherapy. These indicate that GHQ did not decline during the 12 months of treatment with either olaparib or placebo and improved slightly in both groups between 12 and 24 months. A clinically meaningful difference in GHQ would be greater than 10 points, and the difference between the treatment arms is clinically insignificant.

Legend: Mean response of EORTC QLQ-C30 GHQ score over time by treatment group. Panel A: patients who have completed neoadjuvant chemotherapy. Panel B: patients who have completed adjuvant chemotherapy. GHQ score ranges from 0 to 100, higher score indicates better QOL. Adjusted least-square mean responses and 95% CI for time points other than baseline are obtained from mixed model for repeated measures analysis of the GHQ score. The model includes treatment, time and treatment by time interaction, corresponding baseline score, and the baseline score by time interaction. Mean and 95% CI at baseline are based on the raw data.

### FIGURE S6: KM PLOTS FOR IDFS IN THE MATURE COHORT

CI denotes confidence interval.
*Stratified Cox proportional hazards model.
† Kaplan-Meier estimates.

### 5. SUPPLEMENTARY TABLES

TABLE S1
PATIENTS RANDOMIZED IN OLYMPIA, BY COUNTRY

| | | | Placebo | | | |
|---|---|---|---|---|---|---|
| | | | (N = 915) | | | |
| | Olaparib | | no. of | | Total | |
| Country | (N = 921) | | patients (%) | | (N = 1836) | |
| Argentina | 16 | (1.7) | 12 | (1.3) | 28 | (1.5) |
| Australia | 30 | (3.3) | 30 | (3.3) | 60 | (3.3) |
| Austria | 28 | (3.0) | 25 | (2.7) | 53 | (2.9) |
| Belgium | 12 | (1.3) | 26 | (2.8) | 38 | (2.1) |
| Canada | 11 | (1.2) | 23 | (2.5) | 34 | (1.9) |
| China | 117 | (12.7) | 130 | (14.2) | 247 | (13.5) |
| France | 77 | (8.4) | 65 | (7.1) | 142 | (7.7) |
| Germany | 106 | (11.5) | 92 | (10.1) | 198 | (10.8) |
| Hungary | 8 | (0.9) | 9 | (1.0) | 17 | (0.9) |
| Iceland | 5 | (0.5) | 1 | (0.1) | 6 | (0.3) |
| Israel | 30 | (3.3) | 35 | (3.8) | 65 | (3.5) |
| Italy | 30 | (3.3) | 27 | (3.0) | 57 | (3.1) |
| Japan | 64 | (6.9) | 76 | (8.3) | 140 | (7.6) |
| Korea (Republic of) | 53 | (5.8) | 44 | (4.8) | 97 | (5.3) |
| Netherlands | 11 | (1.2) | 18 | (2.0) | 29 | (1.6) |
| Poland | 50 | (5.4) | 59 | (6.4) | 109 | (5.9) |
| Portugal | 7 | (0.8) | 6 | (0.7) | 13 | (0.7) |
| Spain | 63 | (6.8) | 46 | (5.0) | 109 | (5.9) |
| Sweden | 20 | (2.2) | 15 | (1.6) | 35 | (1.9) |
| Switzerland | 4 | (0.4) | 17 | (1.9) | 21 | (1.1) |
| Taiwan, Province of | 8 | (0.9) | 4 | (0.4) | 12 | (0.7) |
| China | | | | | | |
| United Kingdom of | 60 | (6.5) | 46 | (5.0) | 106 | (5.8) |
| Great Britain and | | | | | | |
| Northern Ireland | | | | | | |
| United States of | 111 | (12.1) | 109 | (11.9) | 220 | (12.0) |
| America | | | | | | |

TABLE S2
BRCA1/2 VARIANT STATUS ANALYSED LOCALLY
AND/OR CENTRALLY AT MYRIAD GENETICS

| | | |
|---|---|---|
| Olaparib 300 mg | Placebo | |
| bd | (N = 915) | Overall |
| (N = 921) | no. of patients (%) | (N = 1836) |

Local germline BRCA1 or
BRCA2 status [2]

| gBRCA-P/LP variant | 679 | (73.7) | 680 | (74.3) | 1359 | (74.0) |
|---|---|---|---|---|---|---|
| Variant of Uncertain | 1 | (0.1) | 1 | (0.1) | 2 | (0.1) |
| Significance (VUS) | | | | | | |
| No variant | 0 | (0.0) | 0 | (0.0) | 0 | (0.0) |
| No local result available | 241 | (26.2) | 234 | (25.6) | 475 | (25.9) |
| BRCA1 | | | | | | |
| gBRCA-P/LP variant | 490 | (53.2) | 508 | (55.5) | 998 | (54.4) |
| Variant of Uncertain | 0 | (0.0) | 1 | (0.1) | 1 | (0.1) |
| Significance (VUS) | | | | | | |
| BRCA2 | | | | | | |
| gBRCA-P/LP variant | 188 | (20.4) | 168 | (18.4) | 356 | (19.4) |
| Variant of Uncertain | 1 | (0.1) | 0 | (0.0) | 1 | (0.1) |
| Significance (VUS) | | | | | | |
| BRCA1 & BRCA2 | | | | | | |
| gBRCA1-P/LP variant+ | 1 | (0.1) | 4 | (0.4) | 5 | (0.3) |
| gBRCA2-P/LP variant | | | | | | |
| Central Myriad germline BRCA1 | | | | | | |
| or BRCA2 status [3] | | | | | | |
| gBRCA-D/SD-variant | 777 | (84.4) | 762 | (83.3) | 1539 | (83.8) |
| Variant of Uncertain Significance | 12 | (1.3) | 8 | (0.9) | 20 | (1.1) |
| (VUS) | | | | | | |
| No variant | 1 | (0.1) | 4 | (0.4) | 5 | (0.3) |
| No central Myriad result available | 131 | (14.2) | 141 | (15.4) | 272 | (14.8) |
| [4] | | | | | | |
| BRCA1 | | | | | | |
| gBRCA1-D/SD-variant | 552 | (59.9) | 553 | (60.4) | 1105 | (60.2) |
| Variant of Uncertain Significance | 6 | (0.7) | 5 | (0.5) | 11 | (0.6) |
| (VUS) | | | | | | |
| BRCA2 | | | | | | |
| gBRCA2-D/SD-variant | 224 | (24.3) | 206 | (22.5) | 430 | (23.4) |
| Variant of Uncertain Significance | 6 | (0.7) | 3 | (0.3) | 9 | (0.5) |
| (VUS) | | | | | | |
| BRCA1 & BRCA2 | | | | | | |
| gBRCA1-D/SD-variant + gBRCA2- | 1 | (0.1) | 3 | (0.3) | 4 | (0.2) |

D/SD-variant

[1] Local results include BGI Genomics results for China. Central testing was done by Myriad. OlympiA eligibility required either local results considered Pathogenic (P)/Likely Pathogenic (LP) variants, as now reported by convention in cancer genetics, or Myriad central laboratory results reported as Deleterious (D)/Suspected Deleterious (SD) for the same variant status.
[2] Local BRCA results are available only for patients for whose germline BRCA1 or BRCA2 variant status was known prior to study entry. Central Myriad results are not available for 247 patients enrolled from China. For countries other than China, central Myriad results are available for 1564 of the 1589 patients (98.4%) (see Supplementary Appendix FIG. S2).
[3] Result of confirmatory test carried out centrally by Myriad.
[4] Includes 246 patients randomized in China (olaparib, n = 117, placebo, n = 129) whose local result from BGI Genomics in China confirmed gBRCA-P/LP-variant that meets study eligibility criteria and 1 patient screened in China with a variant of uncertain significance in the placebo arm. Also includes 25 patients from other countries (olaparib, n = 14, placebo, n = 11) tested locally with eligible gBRCA1- or gBRCA2-P/LP-variants for whom central Myriad results are not available, 2 of whom (olaparib, n = 1; placebo, n = 1) have neither local nor central Myriad P/LP variant.

TABLE S3
DISCORDANT LOCAL BRCA STATUS VS CENTRAL MYRIAD BRCA STATUS
FOR 22 (2.0%) PATIENTS AMONG THE 1090 PATIENTS WITH
BOTH LOCAL AND CENTRAL MYRIAD RESULTS AVAILABLE

| | | Central Myriad germline BRCA1 or BRCA2 | | | |
|---|---|---|---|---|---|
| | | status no. of patients (%) | | | |
| | | | Variant of | | |
| | Local germline | | Uncertain | | |
| | BRCA1 or BRCA2 | gBRCA D/SD | Significance | | |
| Overall | status | variant | (VUS) | | No variant |
| Olaparib 300 mg | gBRCA-P/LP | N/A | 10 | (1.8) | 1 (0.2) |
| bd (N = 550) | variant | | | | |
| | Variant of Uncertain | 1 (0.2) | N/A | | 0 (0.0) |
| | Significance (VUS) | | | | |
| | No variant | 0 (0.0) | 0 | (0.0) | N/A |
| Placebo (N = 540) | gBRCA-P/LP | N/A | 7 | (1.3) | 3 (0.6) |
| | variant | | | | |
| | Variant of Uncertain | 0 (0.0) | 0 | (0.0) | N/A |
| | Significance (VUS) | | | | |
| | No variant | 0 (0.0) | N/A | | 0 (0.0) |
| Total (N = 1090) | gBRCA-P/LP | N/A | 17 | (1.6) | 4 (0.4) |
| | variant | | | | |
| | Variant of Uncertain | 1 (0.1) | N/A | | 0 (0.0) |
| | Significance (VUS) | | | | |
| | No variant | 0 (0.0) | 0 | (0.0) | N/A |

[1] Local results include BGI Genomics results for China; central testing was done by Myriad. Percentages presented are based on those for whom both local results and central Myriad results are available. (See FIG. S2 in this Supplementary Appendix)

TABLE S4
CENTRAL RECEPTOR STATUS EXCLUDING CHINESE PATIENTS

| | Olaparib 300 mg | | |
|---|---|---|---|
| | bd | Placebo | Overall |
| | (N = 921) | (N = 915) | (N = 1836) |
| | no. of patients (%) | | |
| Patients with central | 781 | 767 | 1548 |
| pathology results | | | |
| HER2 IHC results | | | |
| 0 | 661 (84.6) | 652 (85.0) | 1313 (84.8) |
| 1+ | 64 (8.2) | 57 (7.4) | 121 (7.8) |
| 2+ | 16 (2.0) | 12 (1.6) | 28 (1.8) |
| 3+ | 0 (0.0) | 2 (0.3) | 2 (0.1) |
| Not interpretable | 0 (0.0) | 0 (0.0) | 0 (0.0) |
| Missing | 40 (5.1) | 44 (5.7) | 84 (5.4) |
| HER2 ISH results [1] | | | |
| Amplified | 1 (0.1) | 3 (0.4) | 4 (0.3) |
| Equivocal | 0 (0.0) | 0 (0.0) | 0 (0.0) |
| Not amplified | 15 (1.9) | 11 (1.4) | 26 (1.7) |
| Not interpretable | 0 (0.0) | 0 (0.0) | 0 (0.0) |
| Missing | 40 (5.1) | 44 (5.7) | 84 (5.4) |
| Hormone Receptor status | | | |
| Positive | 169 (21.6) | 177 (23.1) | 346 (22.4) |
| Negative | 563 (72.1) | 543 (70.8) | 1106 (71.4) |
| Missing | 49 (6.3) | 47 (6.1) | 96 (6.2) |
| ER status | | | |
| Positive | 149 (19.1) | 156 (20.3) | 305 (19.7) |
| Negative | 591 (75.7) | 571 (74.4) | 1162 (75.1) |
| Missing | 41 (5.2) | 40 (5.2) | 81 (5.2) |
| PgR status | | | |
| Positive | 118 (15.1) | 115 (15.0) | 233 (15.1) |
| Negative | 616 (78.9) | 604 (78.7) | 1220 (78.8) |
| Missing | 47 (6.0) | 48 (6.3) | 95 (6.1) |

Percentages based on those with central pathology results. Central pathology review was performed at the European Institute of Oncology (IEO) in Milan, Italy.
HR+ is defined as ER positive and/or PgR positive, where positive is defined as ≥ 1% of cells stained positive.
Missing includes status ‘not done’, ‘unknown’ or ‘missing’.
[1] Only reported for those that are not IHC 0 or 1+

TABLE S5
LOCAL VS CENTRAL LABORATORY RESULTS:
HORMONE RECEPTOR STATUS

| | | Central status[1] | | |
|---|---|---|---|---|
| | Local | HR(+) | HR(−) | Missing [2] |
| | Status | no. of patients (%) | | |
| Olaparib 300 mg bd | HR(+) | 121 (13.1) | 25 (2.7) | 22 (2.4) |
| (N = 921) | HR(−) | 48 (5.2) | 538 (58.4) | 167 (18.1) |
| Placebo (N = 915) | HR(+) | 119 (13.0) | 16 (1.7) | 23 (2.5) |
| | HR(−) | 58 (6.3) | 527 (57.6) | 172 (18.8) |
| Overall (N = 1836) | HR(+) | 240 (13.1) | 41 (2.2) | 45 (2.5) |
| | HR(−) | 106 (5.8) | 1065 (58.0) | 339 (18.5) |

HR+ is defined as ER positive (≥1%) and/or PgR positive (≥1%).
[1] Central laboratory review was not possible for patients recruited in China. Central pathology review was performed at the European Institute of Oncology (IEO) in Milan, Italy.
[2] Missing includes HR status ‘unknown’ or ‘missing’, as well as all patients from China.
Of the 1452 patients that have both a central and a local hormone receptor status, 147 (10%) have discordant results.

TABLE S6
DEMOGRAPHIC AND BASELINE DISEASE CHARACTERISTICS OF THE PATIENTS

| | Olaparib Group | | Placebo Group | | Overall | |
|---|---|---|---|---|---|---|
| Characteristic | (N = 921) | | (N = 915) | | (N = 1836) | |
| Age - median (IQR) | 42 | (36-49) | 43 | (36-50) | 43 | (36-50) |
| Female - no. of patients (%) | 919 | (99.8) | 911 | (99.6) | 1830 | (99.7) |
| Male - no. of patients (%) | 2 | (0.2) | 4 | (0.4) | 6 | (0.3) |
| BRCA gene - no. of patients (%)[1] | | | | | | |
| BRCA1 | 657 | (71.3) | 670 | (73.2) | 1327 | (72.3) |
| BRCA2 | 26 | (28.3) | 239 | (26.1) | 500 | (27.2) |
| BRCA1 & BRCA2 | 2 | (0.2) | 5 | (0.5) | 7 | (0.4) |
| Missing | 1 | (0.1) | 1 | (0.1) | 2 | (0.1) |
| Local or central Myriad BRCA1 or | 920 | (99.9) | 915 | (100) | 1835 | (99.9) |
| BRCA2 germline testing result available [1] | | | | | | |
| Local or central Myriad BRCA1 or | 918 | (99.7) | 912 | (99.7) | 1830 | (99.7) |
| BRCA2 P/LP variant [2] | | | | | | |
| Local testing only [3] | 130 | (14.1) | 141 | (15.4) | 271 | (14.8) |
| Central Myriad testing only | 240 | (26.0) | 234 | (25.6) | 474 | (25.8) |
| No local or central Myriad testing | 1 | (0.1) | 0 | (0.0) | 1 | (0.1) |
| available | | | | | | |
| Local and central BRCA result [4] | 550 | (59.7) | 540 | (59.0) | 1090 | (59.4) |
| Local (+)/Central (+) | 538/550 | (97.8) | 530/540 | (98.1) | 1068/1090 | (98.0) |
| Local (−)/Central (+) | 1/550 | (0.2) | 0/540 | (0.0) | 1/1090 | (0.1) |
| Local (+)/central (−) | 11/550 | (2.0) | 10/540 | (1.9) | 21/1090 | (1.9) |
| Race - no. of patients (%) | | | | | | |
| White | 626 | (68.0) | 599 | (65.5) | 1225 | (66.7) |
| Black/African-American | 19 | (2.1) | 29 | (3.2) | 48 | (2.6) |
| Asian | 259 | (28.1) | 272 | (29.7) | 531 | (28.9) |
| Other | 17 | (1.8) | 15 | (1.6) | 32 | (1.7) |
| Ethnicity - no. of patients (%) | | | | | | |
| Hispanic or Latino | 34 | (3.7) | 24 | (2.6) | 58 | (3.2) |
| Not Hispanic or Latino | 805 | (87.4) | 812 | (88.7) | 1617 | (88.1) |
| Not known, not recorded or refused | 82 | (8.9) | 79 | (8.6) | 161 | (8.8) |
| Jewish descent - no. of patients (%) [5] | | | | | | |
| Yes, of Ashkenazi descent | 41 | (4.5) | 36 | (3.9) | 77 | (4.2) |
| Not of Ashkenazi descent | 880 | (95.5) | 876 | (95.7) | 1756 | (95.6) |
| Geographic region - no. of patients (%) | | | | | | |
| North America | 122 | (13.2) | 132 | (14.4) | 254 | (13.8) |
| South America | 16 | (1.7) | 12 | (1.3) | 28 | (1.5) |
| Europe | 481 | (52.2) | 452 | (49.4) | 933 | (50.8) |
| Asia Pacific and South Africa | 302 | (32.8) | 319 | (34.9) | 621 | (33.8) |
| Prior Neo/Adjuvant chemotherapy - no. | | | | | | |
| of patients (%) | | | | | | |
| Adjuvant | 461 | (50.1) | 455 | (49.7) | 916 | (49.9) |
| Neoadjuvant | 460 | (49.9) | 460 | (50.3) | 920 | (50.1) |
| Anthracycline and taxane regimen | 871 | (94.6) | 849 | (92.8) | 1720 | (93.7) |
| Anthracycline regimen (without | 7 | (0.8) | 13 | (1.4) | 20 | (1.1) |
| taxane) | | | | | | |
| Taxane regimen (without | 43 | (4.7) | 52 | (5.7) | 95 | (5.2) |
| anthracycline) | | | | | | |
| Regimen not reported | 0 | (0.0) | 1 | (0.1) | 1 | (0.1) |
| Less than 6 cycles (neo)adjuvant | 7 | (0.8) | 15 | (1.6) | 22 | (1.2) |
| chemotherapy | | | | | | |
| Neo/Adjuvant platinum therapy - | | | | | | |
| no. of patients (%) | | | | | | |
| No | 674 | (73.2) | 676 | (73.9) | 1350 | (73.5) |
| Yes | 247 | (26.8) | 239 | (26.1) | 486 | (26.5) |
| Concurrent hormone therapy (hormone | 146/168 | (86.9) | 142/157 | (90.4) | 288/325 | (88.6) |
| receptor positive only) - no. of patients | | | | | | |
| (%) | | | | | | |
| Grade - no. of patients (%) [6] | | | | | | |
| Gx: Cannot be assessed | 11/714 | (1.5) | 7/720 | (1.0) | 18/1434 | (1.3) |
| G1: Well differentiated | 2/714 | (0.3) | 3/720 | (0.4) | 5/1434 | (0.3) |
| G2: Moderately differentiated | 128/714 | (17.9) | 114/720 | (15.8) | 242/1434 | (16.9) |
| G3: Poorly differentiated/ | 562/714 | (78.7) | 582/720 | (80.8) | 1144/1434 | (79.8) |
| undifferentiated | | | | | | |
| Not done | 11/714 | (1.5) | 14/720 | (1.9) | 25/1434 | (1.7) |
| Pathological AJCC stage (adjuvant | | | | | | |
| chemotherapy only) - no. of patients (%) | | | | | | |
| 0 | 0/461 | (0.0) | 0/455 | (0.0) | 0/916 | (0.0) |
| IA [7] | 5/461 | (1.1) | 2/455 | (0.4) | 7/916 | (0.8) |
| IB | 15/461 | (3.3) | 11/455 | (2.4) | 26/916 | (2.8) |
| IIA | 264/461 | (57.3) | 250/455 | (54.9) | 514/916 | (56.1) |
| IIB | 70/461 | (15.2) | 75/455 | (16.5) | 145/916 | (15.8) |
| IIIA | 73/461 | (15.8) | 70/455 | (15.4) | 143/916 | (15.6) |
| IIIB | 0/461 | (0.0) | 2/455 | (0.4) | 2/916 | (0.2) |
| IIIC | 28/461 | (6.1) | 41/455 | (9.0) | 69/916 | (7.5) |
| NA [8] | 6/461 | (1.3) | 4/455 | (0.9) | 10/916 | (1.1) |
| CPS + EG score (neo adjuvant | | | | | | |
| chemotherapy only) no. of patients (%) | | | | | | |
| CPS + EG score of 2, 3 or 4 | 398/460 | (86.5) | 387/460 | (84.1) | 785/920 | (85.3) |
| CPS + EG score of 5 or 6 | 22/460 | (4.8) | 15/460 | (3.3) | 37/920 | (4.0) |
| HR+/HER2− | | | | | | |
| CPS + EG score ≤2 [7] | 13/460 | (2.8) | 6/460 | (1.3) | 19/920 | (2.1) |
| CPS + EG score of 3 or 4 | 88/460 | (19.1) | 85/460 | (18.5) | 173/920 | (18.8) |
| CPS + EG score of 5 or 6 | 3/460 | (0.7) | 1/460 | (0.2) | 4/920 | (0.4) |
| Not recorded | 0/460 | (0.0) | 0/460 | (0.0) | 0/920 | (0.0) |
| Triple Negative Breast Cancer | | | | | | |
| CPS + EG score ≤2 | 151/460 | (32.8) | 144/460 | (31.3) | 295/920 | (32.1) |
| CPS + EG score of 3 or 4 | 179/460 | (38.9) | 197/460 | (42.8) | 376/920 | (40.9) |
| CPS + EG score of 5 or 6 | 19/460 | (4.1) | 14/460 | (3.0) | 33/920 | (3.6) |
| Not recorded | 7/460 | (1.5) | 13/460 | (2.8) | 20/920 | (2.2) |
| Hormone receptor status - no. of | | | | | | |
| patients (%) [9] | | | | | | |
| Hormone receptor+/HER2− [10] | 168 | (18.2) | 157 | (17.2) | 325 | (17.7) |
| Triple Negative Breast Cancer [11] | 751 | (81.5) | 758 | (82.8) | 1509 | (82.2) |
| Menopausal status (females only) - no. | | | | | | |
| of patients (%) | | | | | | |
| Premenopausal | 572/919 | (62.2) | 553/911 | (60.7) | 1125/1830 | (61.5) |
| Postmenopausal | 347/919 | (37.8) | 358/911 | (39.3) | 705/1830 | (38.5) |
| Bilateral invasive breast cancer - no. of | | | | | | |
| patients (%) | | | | | | |
| No | 881 | (95.7) | 888 | (97.0) | 1769 | (96.4) |
| Yes | 40 | (4.3) | 27 | (3.0) | 67 | (3.6) |
| Primary breast cancer surgery - no. of | | | | | | |
| patients (%) | | | | | | |
| Mastectomy | 698 | (75.8) | 673 | (73.6) | 1371 | (74.7) |
| Conservative surgery only | 223 | (24.2) | 240 | (26.2) | 463 | (25.2) |
| Missing | 0 | (0.0) | 2 | (0.2) | 2 | (0.1) |
| Local therapy for primary breast cancer - | | | | | | |
| no. of patients (%) | | | | | | |
| Mastectomy plus radiation therapy | 426 | (46.3) | 410 | (44.8) | 836 | (45.5) |
| Mastectomy without radiation | 272 | (29.5) | 263 | (28.7) | 535 | (29.1) |
| therapy | | | | | | |
| Conservative surgery plus | 215 | (23.3) | 231 | (25.2) | 446 | (24.3) |
| radiationtherapy | | | | | | |
| Conservative surgery without | 8 | (0.9) | 9 | (1.0) | 17 | (0.9) |
| radiation | | | | | | |
| therapy | | | | | | |
| Missing | 0 | (0.0) | 2 | (0.2) | 2 | (0.1) |
| Bilateral mastectomy prior to | 332 | (36.0) | 317 | (34.6) | 649 | (35.3) |
| randomisation - no. of patients (%) | | | | | | |
| Bilateral mastectomy after | 98 | (10.6) | 108 | (11.8) | 206 | (11.2) |
| randomisation - no. of patients (%) | | | | | | |
| Bilateral oophorectomy and/or | 185 | (20.1) | 166 | (18.1) | 351 | (19.1) |
| salpingectomy prior to randomisation - | | | | | | |
| no. of patients (%) | | | | | | |
| Bilateral oophorectomy and/or | 375 | (40.7) | 386 | (42.2) | 761 | (41.4) |
| salpingectomy after randomisation - no. | | | | | | |
| of patients (%) | | | | | | |

[1] For a detailed description of local and central Myriad BRCA testing in patients enrolled on OlympiA please see FIG. S2 in this Supplementary Appendix.
Variant interpretation by Myriad Genetics (BRCAnalysis) (n = 1561) and BGI Genomics (n = 247) is performed using multiple established databases (e.g., ClinVar, ClinGen, ENIGMA) and published and internal functional and clinical data, compliant with ACMG published guidelines. The 24 P/LP variants from local labs without central Myriad confirmation were confirmed by the OlympiA Genetics Advisory Committee using published databases as above. Discordant data are enumerated.
[2] There are 6 patients with an important protocol deviation reported for no documented gBRCA-P/LP- variant in BRCA1 or BRCA2 (olaparib, n = 3; placebo n = 3) including 5 patients entered (olaparib, n= 2; placebo n = 3) where either the local or central Myriad testing was done, but with no evidence of a gBRCA-P/LP-variant, and 1 patient in the olaparib group where no local or central Myriad result is available. (See Supplementary Appendix FIG. S2).
[3] Includes 246 patients randomized in China (olaparib, n = 117, placebo, n = 129) whose local result from BGI Genomics in China confirmed gBRCA-P/LP-variant that meets study eligibility criteria and 1 patient screened in China with a variant of uncertain significance in the placebo arm all of whom have no central Myriad result available. Also includes 24 patients from other countries (olaparib, n = 13, placebo, n = 11) for whom central Myriad results are not available. (See Supplementary Appendix FIG. S2).
[4] Patients eligible for the trial are those with a gBRCA-P/LP (D/SD)-variant defined by local testing or central Myriad testing. Patients randomised based on a local test result should also have central Myriad testing done. BRCA1 and BRCA2 testing was done by BGI Genomics in China, there are no Myriad results available for these or 25 other patients tested locally only (See Supplementary Appendix FIG. S2).
[5] Not Ashkenazi Jewish can mean that the patient is either Jewish but not Ashkenazi Jewish, not Jewish or descent recorded as unknown.
[6] Includes only those patients receiving neoadjuvant chemotherapy for whom eCRF indicates histological grade was assessed on treatment naive core biopsy and on all patients receiving adjuvant chemotherapy
[7] Reported as protocol deviations.
[8] These include 2 occult BC (placebo, n = 2), 6 pTx (olaparib, n = 4; placebo, n = 2) and 2 pNx (olaparib, n = 2).
[9] Defined by local test results.
[10] The original protocol activated in 2014 was developed for patients with HER2-negative disease but included only patients with TNBC following regulatory review. When hormone-receptor-positive recurrence risk and combination olaparib and endocrine combination safety rationale was accepted by regulators the protocol was amended in 2015 to include patients with high-risk hormone-receptor positive disease and increase the sample size to the current 1800 level (see Protocol History on www.nejm.org). The first patient with hormone-receptor positive disease was enrolled in December 2015.
[11] Triple negative breast cancer was defined in eligibility criteria as: ER and PgR negative defined as IHC nuclear staining <1%. AND HER2 negative (not eligible for anti-HER2 therapy) defined as: IHC 0, 1+ without ISH OR IHC 2+ and ISH non-amplified with ratio less than 2.0 and if reported, average HER2 copy number <4 signals/cells OR ISH non-amplified with ratio less than 2.0 and if reported, average HER2 copy number <4 signals/cells (without IHC)
Two patients are excluded from the summary of the TNBC subset because they do not have confirmed negative HER2 status.

TABLE S7
SITE OF FIRST IDFS EVENT [1]

| | Olaparib 300 | Placebo |
|---|---|---|
| | mg bd (N = 921) | (N = 915) |
| | no. of patients (%) | |
| IDFS events | 106 (11.5) | 178 (19.5) |
| Distant | 72 (7.8) | 120 (13.1) |
| Distant CNS recurrence | 22 (2.4) | 36 (3.9) |
| Brain metastasis | 21 (2.3) | 36 (3.9) |
| Meningitis carcinomatosa | 1 (0.1) | 0 (0.0) |
| Distant excl. CNS recurrence | 50 (5.4) | 84 (9.2) |
| Bone | 5 (0.5) | 14 (1.5) |
| Lymph nodes (other than local or regional) | 5 (0.5) | 9 (1.0) |
| Lung | 16 (1.7) | 34 (3.7) |
| Liver | 20 (2.2) | 23 (2.5) |
| Pleural effusion | 3 (0.3) | 4 (0.4) |
| Other | 1 (0.1) | 0 (0.0) |
| Regional (ipsilateral) recurrence | 6 (0.7) | 14 (1.5) |
| Axillary lymph nodes | 6 (0.7) | 9 (1.0) |
| Supraclavicular lymph nodes | 0 (0.0) | 3 (0.3) |
| Internal mammary lymph nodes | 0 (0.0) | 1 (0.1) |
| Skin or soft tissue within the regional area | 0 (0.0) | 1 (0.1) |
| Local (ipsilateral) recurrence | 7 (0.8) | 11 (1.2) |
| Breast surgical scar | 1 (0.1) | 3 (0.3) |
| Breast | 3 (0.3) | 4 (0.4) |
| Anterior chest wall | 2 (0.2) | 2 (0.2) |
| Skin or soft tissue within the local area | 1 (0.1) | 2 (0.2) |
| Contralateral invasive breast cancer | 8 (0.9) | 12 (1.3) |
| Second primary malignancies | 11 (1.2) | 21 (2.3) |
| Second primary invasive non-breast | 2 (0.2) | 8 (0.9) |
| ovarian/fallopian tube malignancy | | |
| Second primary invasive non-breast | 9 (1.0) | 13 (1.4) |
| non-ovarian malignancies | | |
| Deaths without a prior IDFS event [2] | 2 (0.2) | 0 (0.0) |

[1] If two recurrence events are reported within 2 months of each other this is referred to as a simultaneous event and will be considered as a single event. In this situation the worst case will be taken as the event ‘type’ but the date of recurrence will be the earliest date of the two events. (reference Hudis et al, 2007)
[2] The 2 deaths without a prior IDFS event were a cardiac arrest and cause unknown.

TABLE S8
ALL DEATHS

| | Olaparib 300 mg bd | Placebo |
|---|---|---|
| | (N = 921) | (N = 915) |
| | no. of patients (%) | |
| Total number of deaths | 59 (6.4) | 86 (9.4) |
| Primary cause of death | | |
| Breast cancer | 55 (93.2) | 82 (95.3) |
| Adverse event [1] | 1 (1.7) | 3 (3.5) |
| Other [2] | 3 (5.1) | 1 (1.2) |
| Missing | 0 (0.0) | 0 (0.0) |

[1] Olaparib: Cardiac arrest (n = 1); Placebo: AML (n = 2), Ovarian cancer (n = 1)
[2] Olaparib: Pulmonary embolism (n = 1), Unknown (n = 1), Pneumonia (n = 1); Placebo: Unknown (n = 1)

TABLE S9
RESULTS OF SENSITIVITY ANALYSES

| | |
|---|---|
| Olaparib | Placebo |

Sensitivity analysis of IDFS in confirmed Myriad gBRCA D/SD patients (n = 1539) [1]

| | | |
|---|---|---|
| Number of patients | 777 | 762 |
| Number of events (%) | 89 (11.5) | 163 (21.4) |
| Estimate of hazard ratio | 0.51 | |
| 99.5% CI for hazard ratio | (0.35, 0.73) | |

Sensitivity analysis of DDFS in confirmed Myriad gBRCA D/SD patients (n = 1539) [1]

| | | |
|---|---|---|
| Number of patients | 777 | 762 |
| Any distant recurrence of disease, | 74 (9.5) | 138 (18.1) |
| second primary cancer, or death (%) | | |
| Estimate of hazard ratio | 0.50 | |
| 99.5% CI for hazard ratio | (0.33, 0.75) | |

Sensitivity analysis of OS in confirmed Myriad gBRCA D/SD patients (n = 1539) [1]

| | | |
|---|---|---|
| Number of patients | 777 | 762 |
| Number of deaths (%) | 47 (6.0) | 79 (10.4) |
| Estimate of hazard ratio | 0.58 | |
| 99% CI for hazard ratio | (0.35, 0.92) | |
| Number of deaths deemed attributable | 44 (5.7) | 75 (9.8) |
| to breast cancer | | |

Central pathology review IDFS analysis (n = 1452) [2]

| | | |
|---|---|---|
| Number of patients | 732 | 720 |
| Number of events (%) | 86 (11.7) | 151 (21.0) |
| Estimate of IDFS hazard ratio | 0.54 | |
| 99.5% CI for IDFS hazard ratio | (0.36, 0.78) | |

Unadjusted IDFS analysis (n = 1836) [3]

| | | |
|---|---|---|
| Number of patients | 921 | 915 |
| Number of events (%) | 106 (11.5) | 178 (19.5) |
| Estimate of IDFS hazard ratio | 0.58 | |
| 99.5% CI for hazard ratio | (0.41, 0.82) | |

Restricted mean survival time (RMST) for IDFS (n = 1836) [3]

| | | |
|---|---|---|
| Number of patients | 921 | 915 |
| RMST ratio (olaparib/placebo) [4] | 1.085 | |
| 99.5% CI for RMST ratio | (1.034, 1.139) | |
| Chi-square: p-value | <0.0001 | |

Proportionality test p-value for IDFS (n = 1836)

| | |
|---|---|
| GT test: Identity transformation of time | 0.02 |
| [5] | |
| GT test: Rank transformation of time [6] | 0.02 |

Proportionality test p-value for DDFS (n = 1836)

| | |
|---|---|
| GT test: Identity transformation of time | 0.20 |
| [5] | |
| GT test: Rank transformation of time | 0.10 |
| [6] | |

Proportionality test p-value for OS (n = 1836)

| | |
|---|---|
| GT test: Identity transformation of time | 0.79 |
| [5] | |
| GT test: Rank transformation of time | 0.71 |
| [6] | |

CI, confidence interval
[1] Patients with confirmed Myriad gBRCA-D/SD-variant, excludes 247 patients randomised in China who do not have central Myriad testing available + another 50 patients from other countries who do not have a central confirmed gBRCA-D/SD-variant result.
[2] Includes patients with both central and local hormone receptor results (see Table S5 in this Supplementary Appendix). Excludes 247 from China and 137 from non-Chinese sites. Central pathology review was performed at the European Institute of Oncology (IEO) in Milan, Italy.
[3] Includes entire intention to treat population.
[4] RMST ratio is the RMST for olaparib divided by the RMST for placebo. Numbers greater than 1.0 reflect an increase in the average months free from an IDFS event for olaparib versus placebo-ie. numbers greater than 1.0 favor olaparib. Olaparib significantly increases restricted mean survival time compared with placebo.
[5] Grambsch-Therneau test using untransformed time in the scaled Schoenfeld residual test.
[6] Grambsch-Therneau test using rank transformation of time in the scaled Schoenfeld residual.

TABLE S10
Invasive disease free survival subgroup analysis

| | N | | |
|---|---|---|---|
| | Olaparib/ | Events (%) | Hazard ratio |
| Subgroup | Placebo | Olaparib/Placebo | & 95% CI [1] |
| Overall | 921/915 | 106 (11.5)/178 (19.5) | 0.581 (0.455, 0.737) |
| Prior Chemo | | | |
| Adjuvant | 461/455 | 36 (7.8)/61 (13.4) | 0.601 (0.394, 0.901) |
| Neoadjuvant | 460/460 | 70 (15.2)/117 (25.4) | 0.555 (0.411, 0.745) |
| Prior Platinum | | | |
| Yes | 247/239 | 34 (13.8)/43 (18.0) | 0.773 (0.490, 1.209) |
| No | 674/676 | 72 (10.7)/135 (20.0) | 0.520 (0.389, 0.689) |
| HR status | | | |
| HR+/HER2− [2] | 168/157 | 19 (11.3)/25 (15.9) | 0.701 (0.381, 1.268) |
| TNBC [3] | 751/758 | 87 (11.6)/153 (20.2) | 0.563 (0.431, 0.730) |
| BRCA variant type | | | |
| BRCA1 | 558/558 | 70 (12.5)/126 (22.6) | 0.524 (0.389, 0.699) |
| BRCA2 | 230/209 | 22 (9.6)/38 (18.2) | 0.515 (0.300, 0.862) |
| BRCA1/2 | 1/3 | 0 (0.0)/0 (0.0) | |
| HR status by prior | | | |
| chemotherapy setting | | | |
| HR+/HER2− with | 104/92 | 13 (12.5)/20 (21.7) | 0.521 (0.253, 1.036) |
| neoadjuvant chemotherapy | | | |
| [2] | | | |
| HR+/HER2− with adjuvant | 64/65 | 6 (9.4)/5 (7.7) | 1.357 (0.409, 4.710) |
| chemotherapy [2] | | | |
| TNBC with neoadjuvant | 354/368 | 57 (16.1)/97 (26.4) | 0.571 (0.410, 0.789) |
| chemotherapy [3] | | | |
| TNBC with adjuvant | 397/390 | 30 (7.6)/56 (14.4) | 0.537 (0.341, 0.830) |
| chemotherapy [3] | | | |
| BRCA status by prior | | | |
| platinum therapy setting | | | |
| BRCA1 with prior platinum | 174/179 | 27 (15.5)/35 (19.6) | 0.775 (0.465, 1.276) |
| therapy for current breast | | | |
| cancer | | | |
| BRCA1 with no prior | 384/379 | 43 (11.2)/91 (24.0) | 0.434 (0.299, 0.619) |
| platinum therapy for current | | | |
| breast cancer | | | |
| BRCA2 with prior platinum | 53/40 | 4 (7.5)/8 (20.0) | |
| therapy for current breast | | | |
| cancer | | | |
| BRCA2 with no prior | 177/169 | 18 (10.2)/30 (17.8) | 0.552 (0.302, 0.980) |
| platinum therapy for current | | | |
| breast cancer | | | |
| BRCA1/2 both with prior | 0/1 | 0/0 (0.0) | |
| platinum therapy for current | | | |
| breast cancer | | | |
| BRCA1/2 both with no prior | 1/2 | 0 (0.0)/0 (0.0) | |
| platinum therapy for current | | | |
| breast cancer | | | |
| Prior platinum by Chemo | | | |
| Prior platinum/ACT | 78/70 | 8 (10.3)/4 (5.7) | |
| Prior platinum/NACT | 169/169 | 26 (15.4)/39 (23.1) | 0.657 (0.396, 1.073) |
| No prior platinum/ACT | 383/385 | 28 (7.3)/57 (14.8) | 0.505 (0.317, 0.787) |
| No prior platinum/NACT | 291/291 | 44 (15.1)/78 (26.8) | 0.507 (0.348, 0.730) |
| Prior platinum by HR status | | | |
| Prior platinum/TNBC | 218/216 | 28 (12.8)/40 (18.5) | 0.700 (0.428, 1.129) |
| Prior platinum/HR+/HER2− | 28/23 | 6 (21.4)/3 (13.0) | |
| No prior platinum/TNBC | 533/542 | 59 (11.1)/113 (20.8) | 0.514 (0.373, 0.702) |
| No prior platinum/ | 140/134 | 13 (9.3)/22 (16.4) | 0.553 (0.271, 1.083) |
| HR+/HER2− | | | |
| Type of prior | | | |
| Neoadjuvant/Adjuvant | | | |
| chemotherapy | | | |
| Anthracycline regimen | 7/13 | 0 (0.0)/2 (15.4) | |
| (without taxane) | | | |
| Taxane regimen (without | 43/52 | 5 (11.6)/8 (15.4) | 0.642 (0.194, 1.925) |
| Anthracycline) | | | |
| Anthracycline and taxane | 871/849 | 101 (11.6)/168 (19.8) | 0.578 (0.451, 0.739) |
| regimen | | | |
| Type of breast surgery prior | | | |
| to randomisation | | | |
| Breast conservation [4] | 223/240 | 20 (9.0)/46 (19.2) | 0.458 (0.265, 0.763) |
| Mastectomy [5] | 698/673 | 86 (12.3)/131 (19.5) | 0.511 (0.333, 0.773) |
| Presence of at risk ovarian | | | |
| tissue prior to first dose of | | | |
| treatment | | | |
| No bilateral oophorectomy | 732/739 | 92 (12.6)/140 (18.9) | 0.648 (0.497, 0.841) |
| Bilateral oophorectomy | 189/176 | 14 (7.4)/38 (21.6) | 0.344 (0.180, 0.619) |
| Pathology axillary node (pN) | | | |
| status at surgery in the | | | |
| TNBC adjuvant cohort [6] | | | |
| Node negative | 203/192 | 13 (6.4)/22 (11.5) | 0.609 (0.298, 1.192) |
| Node positive | 174/177 | 15 (8.6)/31 (17.5) | 0.478 (0.251, 0.870) |
| CPS + EG score (for the post | | | |
| neoadjuvant group only)[7] | | | |
| CPS + EG score of 2, 3 or 4 | 398/387 | 55 (13.8)/96 (24.8) | 0.511 (0.365, 0.709) |
| CPS + EG score of 5 or 6 | 22/15 | 11 (50.0)/10 (66.7) | 0.440 (0.185, 1.060) |
| Age at randomisation | | | |
| Age < 50 years | 699/673 | 79 (11.3)/133 (19.8) | 0.555 (0.419, 0.731) |
| Age 50-64 years | 193/210 | 22 (11.4)/41 (19.5) | 0.578 (0.338, 0.959) |
| Age ≥ 65 years | 29/32 | 5 (17.2)/4 (12.5) | |
| Race | | | |
| White | 626/599 | 75 (12.0)/124 (20.7) | 0.554 (0.414, 0.736) |
| Black/African-American | 19/29 | 4 (21.1)/5 (17.2) | |
| Asian | 259/272 | 25 (9.7)/46 (16.9) | 0.587 (0.355, 0.946) |
| Other | 17/15 | 2 (11.8)/3 (20.0) | |
| Ethnicity | | | |
| Hispanic or Latino | 34/24 | 7 (20.6)/7 (29.2) | 0.648 (0.222, 1.893) |
| Not Hispanic or Latino | 805/812 | 88 (10.9)/153 (18.8) | 0.575 (0.441, 0.746) |
| Not known, not recorded or | 82/79 | 11 (13.4)/18 (22.8) | 0.514 (0.235, 1.074) |
| refused | | | |
| Jewish descent | | | |
| Yes, of Ashkenazi descent | 41/36 | 6 (14.6)/9 (25.0) | 0.486 (0.163, 1.348) |
| No, not of Ashkenazi | 880/876 | 100 (11.4)/169 (19.3) | 0.582 (0.453, 0.744) |
| descent [8] | | | |
| Primary Study Database | | | |
| Breast International Group | 810/806 | 95 (11.7)/160 (19.9) | 0.583 (0.451, 0.749) |
| (BIG) | | | |
| NRG Oncology (US) | 111/109 | 11 (9.9)/18 (16.5) | 0.566 (0.259, 1.182) |
| Geographic region | | | |
| North America | 122/132 | 11 (9.0)/23 (17.4) | 0.483 (0.226, 0.968) |
| South America | 16/12 | 3 (18.8)/5 (41.7) | |
| Europe | 481/452 | 62 (12.9)/95 (21.0) | 0.592 (0.428, 0.814) |
| Asia Pacific and South | 302/319 | 30 (9.9)/55 (17.2) | 0.586 (0.371, 0.908) |
| Africa | | | |

Hazard ratios are provided only if at least 5 IDFS events have occurred in each of the two treatment groups.
Even without correcting for multiple comparisons none of the tests for heterogeneity reached statistical significance
[1] The Cox model included factors for treatment group, subgroup factor and the treatment-by-subgroup interaction. All patients with non-missing subgroup data were included in the model. A hazard ratio < 1 favors olaparib 300 mg bd. The CI was calculated using a profile likelihood approach. These analyses are not inferential. Statistics are provided only if at least 5 IDFS events have occurred in each of the two treatment groups.
[2] HR+ is defined as ER positive and/or PgR positive.
[3] Two patients are excluded from the summary of the TNBC subset because they do not have locally confirmed negative HER2 status.
[4] Breast conservation defined as partial mastectomy/breast quadrantectomy/breast segmentectomy/breast lumpectomy and breast re-excision of margins.
[5] Mastectomy defined as modified radical mastectomy, radical mastectomy (Halsted) or simple mastectomy, or bilateral mastectomy.
[6] TNBC, adjuvant patients only, with sentinel node sampling or axillary node dissection.
[7] Pre-specified subgroup analysis. Includes patients that received neoadjuvant chemotherapy, whether they had hormone receptor positive or triple negative disease.
[8] Not Ashkenazi Jewish can mean that the patient self identifies as either Jewish but not Ashkenazi Jewish, not Jewish or descent recorded as unknown.

TABLE S11
EXPOSURE TO STUDY TREATMENT (SAFETY ANALYSIS SET)

| | |
|---|---|
| Olaparib 300 mg bd | Placebo |
| (N = 911) | (N = 904) |

Total intended exposure (days) [1]

| | | |
|---|---|---|
| Mean | 306.5 | 322.4 |
| SD | 114.80 | 97.54 |
| Median | 364.0 | 364.0 |
| Min | 1 | 2 |
| Max | 492 | 414 |

Actual treatment exposure (days) [2]

| | | |
|---|---|---|
| Mean | 294.4 | 315.1 |
| SD | 113.90 | 97.59 |
| Median | 350.0 | 358.0 |
| Min | 1 | 2 |
| Max | 420 | 404 |

Number of days on 300 mg treatment bd [3]

| | | |
|---|---|---|
| Mean | 245.2 | 306.3 |
| SD | 141.68 | 107.51 |
| Median | 338.0 | 358.0 |
| Min | 1 | 2 |
| Max | 420 | 404 |

Patients with partial treatment end dates are excluded.
[1] Total intended exposure in days = (last dose date − first dose date + 1); does not take account of dose interruptions.
[2] Actual treatment exposure = intended exposure − total duration of dose interruptions, where intended exposure will be calculated as above.
[3] Number of days on 300 mg olaparib/placebo bd (actual exposure for the assigned starting dose).

TABLE S12
DOSE INTENSITY (SAFETY ANALYSIS SET)

| | |
|---|---|
| Olaparib 300 mg bd | Placebo |
| (N = 911) | (N = 904) |

Relative dose intensity (RDI) [1, 2]

| | | |
|---|---|---|
| No. patients | 910 | 903 |
| Mean | 91.9 | 96.7 |
| SD | 12.57 | 8.12 |
| Median | 99.6 | 100.0 |
| Min | 10 | 38 |
| Q1 | 87 | 97 |
| Q3 | 100 | 100 |
| Max | 103 | 100 |

Percentage intended dose (PID) [1, 3]

| | | |
|---|---|---|
| No. patients | 910 | 903 |
| Mean | 81.1 | 92.0 |
| SD | 27.51 | 17.87 |
| Median | 94.8 | 98.9 |
| Min | 0 | 1 |
| Q1 | 75 | 94 |
| Q3 | 100 | 100 |
| Max | 100 | 100 |

Patients with partial treatment end dates are excluded.
[1] Treatment up to one year or until the date of invasive disease (whichever is earliest).
[2] Relative dose intensity (RDI) is the percentage of the actual total dose delivered relative to the intended total dose through to treatment discontinuation.
[3] Percentage intended dose (PID) is the percentage of the actual total dose delivered relative to the intended total dose through to invasive disease.
Due to the eCRF design, the actual cumulative dose does not capture all missed or forgotten doses within an individual day. This will be recorded as if the patient took a full daily dose, which could lead to an overestimation of RDI and PID.

TABLE S13
OF CUMULATIVE EXPOSURE OVER TIME IN
MONTHS (SAFETY ANALYSIS SET)

| Cumulative | Olaparib 300 mg bd | Placebo |
|---|---|---|
| exposure over | (N = 911) | (N = 904) |
| time (months) [1] | no. of patients (%) | |
| >0 months | 910 (99.9) | 903 (99.9) |
| ≥1 month | 848 (93.1) | 872 (96.5) |
| ≥2 months | 824 (90.5) | 847 (93.7) |
| ≥3 months | 801 (87.9) | 836 (92.5) |
| ≥4 months | 782 (85.8) | 821 (90.8) |
| ≥5 months | 769 (84.4) | 805 (89.0) |
| ≥6 months | 757 (83.1) | 794 (87.8) |
| ≥7 months | 752 (82.5) | 782 (86.5) |
| ≥8 months | 739 (81.1) | 771 (85.3) |
| ≥9 months | 719 (78.9) | 758 (83.8) |
| ≥10 months | 706 (77.5) | 753 (83.3) |
| ≥11 months | 685 (75.2) | 733 (81.1) |

Patients with partial treatment end dates are excluded.
[1] Rows are cumulative and subjects are included if they have taken treatment up to and including that day.

TABLE S14A
BLOOD TRANSFUSIONS (SAFETY ANALYSIS SET)

| | Olaparib 300 mg bd | Placebo |
|---|---|---|
| | (N = 911) | (N = 904) |
| | no. of patients (%) | |
| Patients with at least one blood | 53 (5.8) | 8 (0.9) |
| transfusion | | |
| With ≥ grade 3 anemia on treatment | 42 (4.6) | 2 (0.2) |
| With < grade 3 anemia on treatment | 9 (1.0) | 2 (0.2) |
| No anemia reported on treatment | 2 (0.2) | 4 (0.4) |
| Number of patients with only 1 | 37 (4.1) | 6 (0.7) |
| transfusion | | |
| Number of patients with 2 transfusions | 13 (1.4) | 2 (0.2) |
| Number of patients with 3 transfusions | 2 (0.2) | 0 (0.0) |
| Number of patients with 5 transfusions | 1 (0.1) | 0 (0.0) |

Includes blood transfusions up to and including 30 days following the date of last dose date.

TABLE S14B
BLOOD TRANSFUSIONS OVER TIME (SAFETY ANALYSIS SET)

| | Olaparib 300 mg bd | | Placebo | |
|---|---|---|---|---|
| | (N = 911) | | (N = 904) | |
| | no. of | Total no. of | no. of | Total no. of |
| | patients (%) | transfusions | patients (%) | transfusions |
| Treatment month during | | | | |
| which blood transfusion is | | | | |
| given[1] | | | | |
| Up to month 1 | 2 (0.2) | 2 | 0 (0.0) | 0 |
| >=1-2 months | 2 (0.2) | 2 | 1 (0.1) | 2 |
| >=2-3 months | 21 (2.3) | 22 | 0 (0.0) | 0 |
| >=3-4 months | 8 (0.9) | 10 | 1 (0.1) | 1 |
| >=4-5 months | 5 (0.5) | 5 | 1 (0.1) | 1 |
| >=5-6 months | 7 (0.8) | 8 | 1 (0.1) | 1 |
| >=6-7 months | 4 (0.4) | 4 | 0 (0.0) | 0 |
| >=7-8 months | 8 (0.9) | 8 | 0 (0.0) | 0 |
| >=8-9 months | 3 (0.3) | 3 | 0 (0.0) | 0 |
| >=9-10 months | 2 (0.2) | 2 | 1 (0.1) | 1 |
| >=10-11 months | 3 (0.3) | 3 | 1 (0.1) | 1 |
| >=11 months | 5 (0.5) | 5 | 2 (0.2) | 3 |

Includes blood transfusions up to and including 30 days following the date of last dose date.
[1] Patients with multiple transfusions within the same monthly period are counted once for that period.

TABLE S15
Treatment dose reductions (safety analysis set)[1]

| | Olaparib 300 mg bd | Placebo |
|---|---|---|
| | (N = 911) | (N = 904) |
| Patients with no dose reduction (%) | 683 (75.0) | 857 (94.8) |
| Patients with a dose reduction (%) | 228 (25.0) | 47 (5.2) |
| Total number of dose reductions | 287 | 54 |
| Number of patients with a dose reduction | | |
| 1 dose reduction (%) | 170 (18.7) | 40 (4.4) |
| 2 dose reductions (%) | 57 (6.3) | 7 (0.8) |
| 3 or more dose reductions (%) | 1 (0.1) | 0 (0.0) |
| Reason for reduction [2] | | |
| Adverse event (%) | 222 (24.4) | 35 (3.9) |
| Dosing error (%) | 6 (0.7) | 10 (1.1) |
| Administrative reasons (%) | 2 (0.2) | 1 (0.1) |
| Other (%) | 0 (0.0) | 1 (0.1) |

[1] Dose reductions are based on investigator initiated decisions, reductions due to ‘Subject non-compliance’ are omitted.
[2] Reasons for dose reductions are not mutually exclusive for patients with multiple reductions although are counted only once per category.

TABLE S16
Most common AEs leading to permanent discontinuation
of treatment (safety analysis set)

| | Olaparib 300 mg bd | Placebo |
|---|---|---|
| | (N = 911) | (N = 904) |
| Preferred Term | no. of patients (%) | |
| Any AE leading to permanent | 90 (9.9) | 38 (4.2) |
| discontinuation | | |
| Nausea | 18 (2.0) | 3 (0.3) |
| Anaemia | 16 (1.8) | 0 (0.0) |
| Fatigue | 12 (1.3) | 4 (0.4) |
| Neutrophil count decreased | 9 (1.0) | 1 (0.1) |
| Headache | 7 (0.8) | 2 (0.2) |
| Vomiting | 7 (0.8) | 0 (0.0) |
| White blood cell count | 6 (0.7) | 1 (0.1) |
| decreased | | |
| Dizziness | 2 (0.2) | 3 (0.3) |
| Decreased appetite | 2 (0.2) | 2 (0.2) |
| Diarrhoea | 3 (0.3) | 1 (0.1) |
| Breast cancer | 1 (0.1) | 2 (0.2) |
| Drug hypersensitivity | 3 (0.3) | 0 (0.0) |
| Pruritus | 3 (0.3) | 0 (0.0) |
| Abdominal pain upper | 1 (0.1) | 1 (0.1) |
| Arthralgia | 1 (0.1) | 1 (0.1) |

Table shows the number and percentage of patients with that adverse event
Includes AEs with an onset from date of first dose up to 30 days following date of last dose.

TABLE S17
ANY CONCURRENT HORMONE THERAPY FOR PRIMARY
BREAST CANCER IN THE HR+/HER2− SUBGROUP

| | Olaparib 300 mg | | |
|---|---|---|---|
| | bd | Placebo | Overall |
| | (N = 921) | (N = 915) | (N = 1836) |
| | no. of patients (%) | | |
| All HR+/HER2− patients [1] | 168 (100.0) | 157 (100.0) | 325 (100.0) |
| Any concurrent hormone therapy [2] | 146 (86.9) | 142 (90.4) | 288 (88.6) |
| Endocrine therapy | 146 (86.9) | 142 (90.4) | 288 (88.6) |
| Anti-estrogens | 72 (42.9) | 61 (38.9) | 133 (40.9) |
| Tamoxifen | 72 (42.9) | 59 (37.6) | 131 (40.3) |
| Toremifene | 0 (0.0) | 2 (1.3) | 2 (0.6) |
| Aromatase inhibitors | 83 (49.4) | 85 (54.1) | 168 (51.7) |
| Anastrozole | 25 (14.9) | 30 (19.1) | 55 (16.9) |
| Exemestane | 23 (13.7) | 23 (14.6) | 46 (14.2) |
| Letrozole | 41 (24.4) | 37 (23.6) | 78 (24.0) |
| Pituitary and hypothalamic hormones | 39 (23.2) | 33 (21.0) | 72 (23.7) |
| and analogues | | | |

Each treatment will be counted a maximum of once per patient. Percentages presented are based on those patients that have hormone receptor positive breast cancer.
Of the 325 patients with hormone-receptor positive disease, 147 had oophorectomy either before (n = 74) or following (n = 73) randomization. These numbers for olaparib are: 42, and 33; and for placebo are: 32 and 40.
[1] HR+ is defined as ER positive and/or PgR positive based on a cut-off for positivity of ≥ 1% of cells stained positive.
[2] NB. The protocol defines hormone-receptor positivity as ≥ 1% of cells stained positive but use of adjuvant endocrine therapy was determined by institutional and/or national guidelines, which may not recommend endocrine therapy for patients with tumors with 1-9% staining of cells for estrogen receptor explaining the lack of endocrine therapy use in 11.4% of patients balanced between treatment arms.

TABLE S18
Important protocol deviations
Important protocol deviations (IPD)s are a concise list of pre-defined protocol deviations
which have a very high likelihood of influencing the primary efficacy and/or the secondary
safety results. IPD's are also distinct from simple protocol deviations.

| | Olaparib 300 mg | | |
|---|---|---|---|
| | bd | Placebo | Overall |
| | (N = 921) | (N = 915) | (N = 1836) |
| | no. of patients (%) | | |
| Number of patients with at least one important | 16 (1.7) | 14 (1.5) | 30 (1.6) |
| protocol deviation triggering a sensitivity | | | |
| analysis [1] | | | |
| No histologically confirmed non-metastatic | 3 (0.3) | 0 (0.0) | 3 (0.2) |
| primary invasive adenocarcinoma of the breast | | | |
| [2] | | | |
| No documented germline pathogenic/likely | 3 (0.3) | 3 (0.3) | 6 (0.3) |
| pathogenic variant in BRCA1 or BRCA2 [2] | | | |
| Randomized but did not receive any study | 10 (1.1) | 11 (1.2) | 21 (1.1) |
| treatment [2] | | | |
| Number of patients with at least one important | 130 (14.1) | 122 (13.3) | 252 (13.7) |
| protocol deviation excl. important GCP | | | |
| violations [3] | | | |
| No histologically confirmed non-metastatic | 3 (0.3) | 0 (0.0) | 3 (0.2) |
| primary invasive adenocarcinoma of the breast | | | |
| [2] | | | |
| No documented germline pathogenic/likely | 3 (0.3) | 3 (0.3) | 6 (0.3) |
| pathogenic variant in BRCA1 or BRCA2 [2] | | | |
| Randomized but did not receive any study | 10 (1.1) | 11 (1.2) | 21 (1.1) |
| treatment [2] | | | |
| Not fulfilling criteria for high risk disease | 25 (2.7) | 12 (1.3) | 37 (2.0) |
| Inadequate breast surgery and/or radiotherapy | 7 (0.8) | 8 (0.9) | 15 (0.8) |
| Inadequate axilla surgery | 5 (0.5) | 1 (0.1) | 6 (0.3) |
| Completed less than 6 cycles of neoadjuvant or | 7 (0.8) | 15 (1.6) | 22 (1.2) |
| adjuvant chemotherapy containing | | | |
| anthracyclines, taxanes or the combination of | | | |
| both | | | |
| Peri-operative chemotherapy (patients who had | 4 (0.4) | 6 (0.7) | 10 (0.5) |
| both neoadjuvant and adjuvant therapy; | | | |
| ‘unquantifiable risk of disease relapse’) | | | |
| Evidence of metastatic disease (to include only | 2 (0.2) | 4 (0.4) | 6 (0.3) |
| those patients who had suspicion or | | | |
| confirmation of recurrence prior to | | | |
| randomisation) | | | |
| No staging or insufficient staging | 67 (7.3) | 66 (7.2) | 133 (7.2) |
| Prior PARP inhibitor use | 0 (0.0) | 0 (0.0) | 0 (0.0) |
| Prior cancer < 5 years ago including MDS/t- | 0 (0.0) | 2 (0.2) | 2 (0.1) |
| AML | | | |
| Received no study treatment whatsoever for a | 5 (0.5) | 4 (0.4) | 9 (0.5) |
| period of more than 7 days due to errors in | | | |
| dispensing of medication | | | |
| Received an alternative study treatment to that | 0 (0.0) | 0 (0.0) | 0 (0.0) |
| which they were randomized | | | |
| Received prohibited concomitant medication | 10 (1.1) | 12 (1.3) | 22 (1.2) |
| Received additional anti-cancer therapy prior to | 0 (0.0) | 0 (0.0) | 0 (0.0) |
| IDFS event [4] | | | |
| Received other investigational agent prior to | 0 (0.0) | 0 (0.0) | 0 (0.0) |
| IDFS event | | | |
| Lack of confirmatory exams for events that | 0 (0.0) | 1 (0.1) | 1 (0.1) |
| count towards the analysis end points, efficacy | | | |
| and safety | | | |

[1] Statistical Analysis Plan specified that a sensitivity analysis for primary efficacy be conducted if > 10% of the full analysis set did not have the intended disease or indication or did not receive any study medication. This is shown in Table S9.
[2] An important protocol deviation (IPD) that triggers a sensitivity analysis
[3] The same patient may have had more than one important protocol deviation. Important protocol deviations are those that could have a strong influence on the interpretation of the efficacy or safety results.
[4] Other than hormone therapy or adjuvant bisphosphonates permitted in the protocol.

### 6. REFERENCE

1. Mittendorf EA, Jeruss JS, Tucker SL, et al. Validation of a novel staging system for disease-specific survival in patients with breast cancer treated with neoadjuvant chemotherapy. J Clin Oncol 2011; 29:1956-62.

What is claimed is:

1. A method of preventing, reducing, or delaying the reoccurrence of breast cancer in a subject following local treatment and neoadjuvant or adjuvant chemo therapy, the method comprising:
administering to the subject a therapeutically effective amount of 4-[(3-{[4-(cyclopropane carbonyl)piperazine-1-yl]carbonyl}-4-fluorophenyl)methyl]-2H-phthalazin-1-one (olaparib), or a hydrate, solvate, or prodrug thereof.

2. A method of treating a subject with breast cancer following local treatment and neoadjuvant or adjuvant chemo therapy, said method comprising the adjuvant treatment of the subject with a therapeutically effective amount of 4-[(3-{[4-(cyclopropane-carbonyl)piperazine-1-yl]carbonyl}-4-fluorophenyl)methyl]-2H-phthalazin-1-one (olaparib), or a hydrate, solvate, or prodrug thereof.

3. The method of claim 1 or 2, wherein the subject has one or more germline BRCA1 and/or BRCA2 gene mutations.

4. The method of claim 3 wherein the germline BRCA1 and/or BRCA2 gene mutations are pathogenic or likely pathogenic (gBRCA-P/LP-variant).

5. The method of any one of claims 1 to 4 wherein the breast cancer is HER2-negative breast cancer.

6. The method of claim 5 wherein the breast cancer is HER2-negative early stage (Stage II-III) breast cancer.

7. The method of any one of claims 1 to 6 wherein the local treatment comprises surgery so as to remove breast cancer tissue, and optionally includes radiotherapy

8. The method of any one of claims 1 to 7, wherein the subject has completed at least 6 cycles of neoadjuvant or adjuvant chemotherapy.

9. The method of claim 8, wherein the neoadjuvant or adjuvant chemotherapy contains anthracyclines, taxanes or a combination of both.

10. The method of any one of claims 1 to 9 wherein the therapeutically effective amount of olaparib is 300 mg administered twice daily.

11. The method of claim 10 wherein the therapeutically effective amount of olaparib is 300 mg administered twice daily for a period of 1 year.

12. The method of any one of claims 1 to 11 wherein invasive disease-free survival is improved relative to subjects treated with placebo.

13. The method of claim 12 wherein the probability of invasive disease free survival is about 86% at about 3 years following initiation of olaparib treatment.

14. The method of claim 12 wherein the probability of invasive disease free survival is improved by from about 1 to about 10% at about 3 years following initiation of olaparib treatment, such as from about 1 to about 9% at about 3 years, such as from about 5 to about 9% at about 3 years.

15. 4-[(3-{[4-(cyclopropane carbonyl)piperazine-1-yl]carbonyl}-4-fluorophenyl)methyl]-2H-phthalazin-1-one (olaparib), or a hydrate, solvate, or prodrug thereof for use in (or for use in the manufacture of a medicament for) the adjuvant treatment, after local treatment and neo adjuvant or adjuvant chemotherapy, of a subject having breast cancer.

16. The use of claim 15, wherein the subject has one or more germline BRCA1 and/or BRCA2 gene mutations.

17. The use of claim 16 wherein the germline BRCA1 and/or BRCA2 gene mutations are pathogenic or likely pathogenic (gBRCA-P/LP-variant).

18. The use of any one of claims 15 to 17 wherein the breast cancer is HER2-negative breast cancer.

19. The use of claim 18 wherein the breast cancer is HER2-negative early stage (Stage II-III) breast cancer.

20. The use of any one of claims 15 to 19 wherein the local treatment comprises surgery so as to remove breast cancer tissue, and optionally includes radiotherapy

21. The use of any one of claims 15 to 20, wherein the subject has completed at least 6 cycles of neoadjuvant or adjuvant chemotherapy.

22. The use of claim 21, wherein the neoadjuvant or adjuvant chemotherapy contains anthracyclines, taxanes or a combination of both.

23. The use of any one of claims 15 to 22 wherein the therapeutically effective amount of olaparib is 300 mg administered twice daily.

24. The use of claim 23 wherein the therapeutically effective amount of olaparib is 300 mg administered twice daily for a period of 1 year.

25. The use of any one of claims 15 to 24 wherein invasive disease-free survival is improved relative to subjects treated with placebo.

26. The use of claim 25 wherein the probability of invasive disease free survival is about 86% at about 3 years following initiation of olaparib treatment.

27. The use of claim 25 wherein the probability of invasive disease free survival is improved by from about 1 to about 10% at about 3 years following initiation of olaparib treatment, such as from about 1 to about 9% at about 3 years, such as from about 5 to about 9% at about 3 years.

28. A method of improving invasive disease survival (or overall survival or distant-disease-free survival) by providing adjuvant treatment to a subject with a prior diagnosis of germline mutated BRCA1 and/or BRCA2 breast cancer, said subject having previously had local treatment and neoadjuvant or adjuvant chemotherapy, the method comprising the step of administering to such a subject a therapeutically effective amount of 4-[(3-{[4-(cyclopropane-carbonyl)piperazine-1-yl]carbonyl}-4-fluorophenyl)methyl]-2H-phthalazin-1-one (olaparib), or a hydrate, solvate, or prodrug thereof.

29. The method of claim 28 wherein the breast cancer is HER2-negative breast cancer.

30. The method of claim 29 wherein the breast cancer is HER2-negative early stage (Stage II-III) breast cancer.

31. The method of any one of claims 28 to 30 wherein the local treatment comprises surgery so as to remove breast cancer tissue, and optionally includes radiotherapy.

32. The method of any one of claims 28 to 31, wherein the subject has completed at least 6 cycles of neoadjuvant or adjuvant chemotherapy.

33. The method of claim 32, wherein the neoadjuvant or adjuvant chemotherapy contains anthracyclines, taxanes or a combination of both.

34. The method of any one of claims 28 to 33 wherein the therapeutically effective amount of olaparib is 300 mg administered twice daily.

35. The method of claim 34, wherein the therapeutically effective amount of olaparib is 300 mg administered twice daily for a period of 1 year.

36. The method of any one of claims 28 to 35, wherein invasive disease-free survival is improved relative to subjects treated with placebo.

37. The method of claim 36, wherein the probability of invasive disease free survival is about 86% at about 3 years following initiation of olaparib treatment.

38. The method of claim 36, wherein the probability of invasive disease free survival is improved by from about 1 to about 10% at about 3 years following initiation of olaparib treatment, such as from about 1 to about 9% at about 3 years, such as from about 5 to about 9% at about 3 years.

39. The method of claim 36 wherein, the improvement in invasive disease free survival at three years is about 9%.

40. The method of any one of claims 28 to 35 wherein, wherein distant disease-free survival is improved relative to subjects treated with placebo.

41. The method of claim 40, wherein the improvement in distant disease free survival at about 3 years is up to about 8%, such as up to about 7%, such as from about 1 to about 8%, such as from about 1 to about 7%, such as from about 3% to about 8%, such as from about 3% to about 7%.
